(19) United States
(12) Patent Application Publication
(10) Pub. No.: US 2026/0240667 A1
(43) Pub. Date: Aug. 20, 2026

(54) Apparatus and Method of Fitting a Prosthetic Using Structural Fabrics

(71) Applicant: James Jay Martin, Oklahoma City, OK (US)

(72) Inventor: James Jay Martin, Oklahoma City, OK (US)

(21) Appl. No.: 19/057,866

(22) Filed: Feb. 19, 2025

(51) Int. Cl. A61F 2/78; A61F 2/80

(52) U.S. Cl. CPC A61F 2/7812; A61F 2/80; A61F 2002/7818

(57) ABSTRACT

Disclosed is a prosthetic socket system that uniquely uses conforming fabrics to provide increased comfort and simplicity of fitting, in order to make long-lasting comfortable prosthetics more accessible to the global amputee population. The system comprises modular, conforming panels that interconnect to form a composite flexible inner socket. This inner socket is suspended from a modular exoskeleton support structure that can be adjusted for user-specific sizing. A containment strap system with adjustable webbing straps and an integrated adjustor mechanism provides fine tension control by selectively winding or releasing the webbing to secure the residual limb. The interplay between the limb's outward forces and the inward forces from the tensioned containment system dynamically defines an anatomically specific socket shape, thereby enhancing biomechanical support and user comfort. The system is designed for rapid, on-site fitting without requiring specialized fabrication equipment or highly trained prosthetists.

### CROSS-REFERENCE TO RELATED APPLICATIONS

The present application claims priority to U.S. Provisional Patent Application Ser. No. 63/630,662 filed Feb. 21, 2024 and incorporated by reference herein.

### FIELD OF THE INVENTION

The present invention relates generally to an apparatus, system, and method of fitting a prosthesis for amputees. More particularly, the present invention is a new and improved prosthetic socket apparatus, system, and method utilizing conforming structural fabrics to provide increased comfort to the user, and which does not require a highly-trained prosthetist or conventional fabrication machinery to perform the prosthetic fitting.

### BACKGROUND OF THE INVENTION

There was a time when wooden sockets were common. Fiberglass resins replaced using wood, and carbon fiber ultimately replaced fiberglass. In recent years, 3D printed sockets are becoming an alternative to carbon fiber fabrication methods. The evolution of socket eras has been largely catalyzed by the invention of simpler fabrication methods, but the underlying socket designs have seen very little change for many decades. Wooden sockets are categorically similar to conventional carbon fiber or 3D printed sockets-they each force the dynamic human limb to fit into a contoured, yet static, rigid, and unforgiving bucket.

In many parts of the developing world, there is a gross lack of trained practitioners and specialized equipment needed to fabricate and fit prosthetics, leaving over 40 million amputees without access to even antiquated hard sockets. Even in developed countries like the United States, many amputees do not have time and financial resources available for the costly multi-day fitting and fabrication processes required to make conventional prosthetics.

It is an objective of this invention to make comfort and a restored quality of life accessible to all amputees around the world-regardless of socio-economic status or geographic location.

To accomplish this, there must be a paradigm shift in how prosthetics are fit. There is simply not a logistically viable solution to serve the global amputee population with existing methods of fitting conventional prosthetics. There are not enough Prosthetists in all local villages, communities, cities, and countries of the world, nor is there accessible know-how and training needed to equip enough additional Prosthetists. There is not available equipment needed to make conventional prosthetics in all villages, communities, cities, and countries of the world. And there is not enough time for any given Prosthetist to serve the vast underserved and unserved amputee populations in their local villages, communities, cities, and countries of the world, as it takes considerable time to fit and fabricate a conventional prosthetic to each patient.

While 3D printed socket designs have promised to become the solution to the problem, they still require a Prosthetist, training, expensive equipment (3D printers), and technical know-how that is just not accessible on a level to have any sizable global impact.

There needs to be a prosthetic socket system and method that eliminates the need for traditional and/or expensive fabrication equipment, eliminates the unforgiving rigid socket materials used to make conventional prosthetics, and eliminate the requirement for highly trained and skilled Prosthetists to perform the prosthetic fitting.

To further complicate the scenario, the fortunate global amputee population who does have access to being fit with a prosthesis, typically receives an antiquated ‘conventional’ socket design that is rigid, static, and unforgiving. The human limb however is dynamic and constantly changes in size, volume, and contouring. As a result, the vast majority of amputees struggle with discomfort related issues, as there is a certain level of ongoing asymmetry between the statically-shaped prosthetic socket and the dynamically changing human limb. High point-pressures, rubbing, chaffing, encapsulated heat, pressure sores, and/or a limited quality of life can be experienced by amputees wearing conventional sockets.

### DESCRIPTION OF THE KNOWN PRIOR ART

Nearly every product that is designed to physically interface with the human body offers conforming materials that match to the underlying body shape, such as backpacks, shoes, and car seats. These products are simple to fit and adjust to the body, without the need for highly-skilled practitioners with extensive training. However, the majority of conventional prosthetics remain static, rigid, and unforgiving, as they encapsulate the dynamic human amputated limb with minimally conforming or non-conforming materials. Available prosthetic socket designs also require a highly trained Prosthetist to be able to fit them, and most require costly and complicated conventional fitting and fabrication methods.

In recent years a few prosthetic socket systems have emerged which offer more conformity than their conventional counterpart, however, these other socket designs are limited in their ability to offer the full conformity needed to fully match to the amputated limb, in real-time. They also require a highly trained Prosthetist to fit them, and they require traditional fabrication methods to fabricate them. Each of these limitations results in the inability to serve the global amputee population in any sizable way.

The RevoLimb design uses an adjustable Boa lacing system to slightly tighten or loosen various rigid panels within the socket. This design does not provide any conformity to the limb tissue, is challenging to fabricate using conventional fabrication methods, and requires a highly-trained Prosthetist to fit.

Cornell (U.S. Pat. No. 8,945,237) disclosed a socket design using a fabric panel spanned across one side of a conventional socket frame, referred to as a sail. In his sail design, the limb is fully encapsulated with a combination of traditional rigid frame, and a section of flexible fabric panel. The main advantage that his sail material offers is that it provides more flexibility in sitting for certain portions of the socket, and it is adjustable. However, it remains an encapsulated socket with a large portion of rigid materials, is challenging to fit and fabricate, just as is a full conventional socket design, and requires a Prosthetist to fit.

In Device for mounting an artificial limb to the stump of an amputated limb-US4634446A, The device has a flexible sleeve where the stump would slide into in order to connect the stump to the prosthetic device. The sleeve is made of a material that would not stretch when load is added to it. The goal of this device is to be able to securely fasten the artificial limb to the stump but without the use of the standard rigid components, as this causes discomfort.

Prefabricated therapeutic prosthesis for below-knee amputees-US5529575A, is a below-knee prosthesis for immediate post-operative use includes a shrinker socket and a frame fitting and following the contours of the lower socket into which the shrinker socket can be slid and secured.

Adjustable Prosthetic socket-US20060009860A1 OR US6991657B1, is an adjustable prosthetic socket of the instant invention includes a cup, a lateral wall, and a brim. The cup includes a lateral cutaway portion, a lower closed portion, and an upper open portion. The brim is adapted for adjustable mounting in said cup via said upper open portion while the lateral wall is adapted for adjustable mounting in said lateral cutaway.

Prosthesis Socket and System Comprising a Prosthesis Socket and Prosthesis Device-US20100191348A1 is a sleeve designed for receiving the stump. The shell is essentially one piece that wraps around the leg and the ends overlap. There is a strap attached to the shell that is used for tightening the shell around the leg.

Prosthetic limb having an adjustable socket-WO2015013560A1 is a socket for a prosthetic limb that provides a body extending along an axial direction and defining a cavity configured to receive a residual limb of a user. The socket further comprises an adjustment mechanism attached to the body configured to adjust a volume of the cavity to accommodate an increase or decrease in a volume of the user's residual limb.

In Transtibial prosthetic socket with textile jacket-US10172728B2, A transtibial prosthetic socket frame may include a distal base assembly having a base plate, a carriage configured to support a socket suspension arrangement, and a distal prosthetic component connector. The distal base assembly supports a set of struts that includes two anterior struts and a single posterior strut. The set of struts and distal base assembly collectively define a prosthetic socket cavity having a central longitudinal axis and a residual limb hosting volume. The distal prosthetic component connector has a connecting adapter that is rotatable with respect to the prosthetic socket, and moveable with respect to the base plate between being aligned with the prosthetic socket's central longitudinal axis and a position offset therefrom.

In Post-operative residual limb support-US9855154B2, A post-operative residual limb support assembly includes an upper frame configured to extend about at least a portion of a residual limb; and a lower frame coupled to the upper frame and configured to receive an end of the residual limb. The upper frame and the lower frame are adjustable relative to one another in at least one of a linear and rotational manner.

In Transfemoral prosthetic socket with a textile-based cover and intra-frame force applicators-US10179056B2, An intra-frame positioning sling may be disposed internally within a prosthetic socket frame. The sling may include one or more proximal suspension portions adapted to suspend from a proximal aspect of the frame and a tensioning system. The sling may include a first longitudinal side with one or more proximal suspension portions and an opposite longitudinal side that includes the residual limb interfacing or force application portion of the sling. When the tensioning system is tensioned, the sling is pulled toward the first longitudinal side of the prosthetic socket frame. A set of two positioning slings may be rigged in a transfemoral prosthetic socket frame; one sling applies force on a medial side of a hosted residual limb, and the other sling applies force on the lateral side.

In Modular prosthetic devices and prosthesis systems-US11382775B2, An adjustable prosthesis system for a residual limb includes: an adjustable outer shell surrounding the residual limb having a plurality of overlapping flaps, a top opening along a top edge extending around the outer shell, a bottom having a bottom edge opposite the top edge and extending around the bottom, a first side, and a second side having an adjustable width, and a plurality of adjustable overlapping side ends, some of which overlap and some do not, extending from the top edge to the bottom edge; a base adjacent the bottom of the outer shell and connected to it; and a closure component attached to the outer shell to compress the residual limb. Tightening the closure component applies a pulling force to at least one of the first side and the second side, thereby causing a reduction in width of a discontinuity and increased overlap of the side ends.

In Weight-bearing lower extremity brace-US9820870B2, a device and/or system (e.g., redistributing weight away from a subject's foot), which may comprise (a) a platform, (b) at least one vertical support fixed to the platform and extending upwardly from the platform, and (c) at least one cuff (i) configured to surround and releasably grip at least a portion of a subject's leg other than the foot and (ii) mounted (e.g., adjustably) to the at least one vertical support at a vertical position along the at least one vertical support sufficient to suspend a subject's foot in a non-weight-bearing position above the platform during ambulation, wherein the platform, the at least one vertical support, and the at least one cuff together are configured to bear at least the subject's full weight.

In Prosthetic devices, systems including the same, and related methods-US20230255803A1, A prosthetic device for engaging a residual limb includes a plurality of elongate counters configured to receive at least a portion of the residual limb within a volume defined by the plurality of elongate counters. Each respective elongate counter is configured to counteract forces applied to the residual limb via the prosthetic device. The prosthetic device also includes a socket and a selective adjustment system, and may include an independent counter engaged with the socket. The socket is configured to engage with the residual limb, and may include a lower textile socket coupled to the plurality of elongate counters and an upper textile socket coupled to the lower textile socket. The selective adjustment system is configured to secure the prosthetic device to the residual limb and to provide selective adjustment of compression of the socket and the plurality of elongate counters around the residual limb.

In Variable compliance prosthetic socket with breathable matrix-US11672678B2, A socket for engaging a residual limb includes a plurality of hard counters, each configured to engage the residual limb at defined “go points”. The go points can bear pressure without discomfort or impeding range of motion. The counters are further configured to avoid contact with identified “no go” points where excess tissue may inhibit motion or experience irritation upon movement and which include one or more contoured pads to more effectively engage the bony prominences of the limb without inhibiting motion. A chassis from which each of the counters may flexibly depend connects the residual limb mechanically to a limb extension. A lacing system alternately intersects the counters and terminates in a tensioning reel. Rotation of the tensioning reel in a first direction will draw the laces over each of the counters such that the tensioned laces will draw counters together to engage the residual limb.

In a prior invention by Martin (Air hammock for a prosthetic-US20200397602A1, and Method of fitting a prosthetic interface system using compliant members-US20220062014A1), a modular and conforming socket system was disclosed. In this design, a combination of modular parts could be assembled around the limb to generally match to the underlying limb shape. The majority of these parts were made of plastics, metals, and custom fabricated carbon fiber materials which were assembled together using truss nuts-similar to an Erector Set. A small portion of the brim of its above knee socket uses a fabric strap that spans between either two struts, and/or custom molded carbon fiber laminated sections, and which spans as one webbing piece from posterior-lateral through the medial side to the anterior-medial side of an above knee limb.

The Martin disclosure requires a trained Prosthetist to fit in practical real-world application. There are also a number of biomechanical differences between the prior Martin disclosures and this disclosure, including:
1. The prior Martin disclosures use a combination of anchored struts and floating struts around the limb.
   1.1. This disclosure uses only anchored struts around the limb, in order to provide increased biomechanical control, in coordination with the assembly configuration.
2. The prior Martin disclosures subcomponents connect together using Truss Nuts, screws, and thin plastic cross connectors.
   2.1. This disclosure may use a combination of various sections of structural fabrics which may self-connect together in a manner similar to velcro or other similar materials, without requiring rigid connectors like truss nuts or screws.
3. The prior Martin disclosures have an open un-encapsulated design, with minimal surface area in contact with the limb, therefore causing high point pressures in isolated areas over the limb.
   3.1. This disclosure may use structural fabrics, including the containment straps, to largely encapsulate the limb, thereby distributing the forces about a larger surface area and minimize point pressures.
4. The prior Martin disclosure BK design tightens around the limb by drawing in the posterior lateral and posterior medial struts toward each other.
   4.1. This disclosure's BK may tighten around the limb by drawing in the medial and lateral sides, through tightening the anterior to posterior dimension of the socket, providing more of a circumferential tightening effect, though in a triangular form factor.
5. The prior Martin disclosure for AK tightens around the limb by drawing in the anterior only, between the lateral panel or strut and the anterior medial strut.
   5.1. This disclosure's AK may tighten around the limb by drawing in the anterior and posterior sides together, through tightening the medial to lateral dimension of the socket about the limb, providing more of a circumferential tightening effect, though in a triangular form factor.
6. The prior Martin disclosure tightens by using ratchet and ladder straps, which are anchored to the frame structure on both ends, therefore the tightening only occurs via the relative length difference between the two anchored ends of the adjustor mechanism.
   6.1.This disclosure may tighten via a unique self-designed webbing strap adjustor mechanism. Neither end of the straps need be affixedly anchored to the frame but may rather float with non-anchored points. Therefore the relative tightening of the strap may occur circumferentially around the limb, or around portions of the limb, and the related structural fabric panels may cause only certain areas of the socket to tighten due to the structural fabric's characteristics.
7. The prior Martin disclosures use anchored narrow plastic Air Hammock Petals to disperse forces under the struts.
   7.1. This disclosure doesn't disperse forces by an anchored element to a Strut but rather may disperse the force through structural fabrics only.
8. The prior Martin disclosures use a custom molded lamination to generate the alignment between the limb and the prosthetic knee and/or foot.
   8.1. This disclosure may use a uniquely modular base to generate the alignment, along with off-the-shelf conventional alignable prosthetic components, and does not require a custom molded lamination or fabrication thereof.
9. The prior Martin disclosure AK uses a continuous fabric brim that is sewn to length on both ends.
   9.1. This disclosure may use multiple adjustable straps, in coordination with fabric panels, with no webbing fabric connected in the medial brim area. Further, the straps may be connected to structural fabric panels, whose contouring may be morphed to the limb shape in real-time based on the tightness of the containment strap system.
10. The prior Martin disclosure BK uses a custom molded lamination, plastic cross connectors, and a straight fabric strap to form the brim.
   10.1. This disclosure may use a multiple fabric straps in a V-shape to form the posterior brim, and structural fabrics to form the anterior brim, without the need for a custom molded lamination. Further, the straps may be connected to structural fabric panels, whose contouring may be morphed to the limb shape in real-time based on the tightness of the containment strap system.
11. The prior Martin disclosure BK posterior brim can not fold over during range of motion, and therefore presses into the back of the knee during knee flexion range of motion.
   11.1. This disclosure's BK brim, being entirely made of soft, conforming fabric, may fold around 90 degrees during knee flexion, but remains supportive during stance phase.
12. The prior Martin disclosure circumferential fit is determined by hard-mounted plastic Cross Connectors and plastic ratchet and ladder straps to a determined length.
   12.1.This disclosure's circumferential fit may be determined by floating fabric webbing containment straps made of conforming structural fabric materials, and which do not need to be anchored to any structure.
13. The prior Martin disclosure uses mounted buckles to connect to brim webbing elements to the frame structure.
   13.1. This disclosure may use non-mounted, floating buckles to connect webbing elements to each other, but not to any structure.
14. The prior Martin disclosure distal contouring is accomplished by forming a custom molded laminated distal end, or a pre-formed plastic element referred to as the Air Hammock Distal Receptacle.
   14.1. This disclosure's distal contouring may be accomplished by forming velcro-compatible, or similar, structural fabric sub-components together into a bowl shape that match to the specific limb shape in real-time, without the need for traditional fabrication methods.
15. The entire prior Martin disclosure assembly is one solidly connected assembly, and is not machine washable.
   15.1. This disclosure may have a separable fabric inner socket that can be easily removed from the frame structure for washing in a washing machine.
16. The prior Martin disclosure is predominantly made of plastic parts.
   16.1. This disclosure may be predominantly made of fabric parts that may be sewn together and/or adhered by their velcro-compatible characteristics.
17. The prior Martin disclosure BK requires custom laminated ears to be formed around the sides of the knee shape for stability.
   17.1. This disclosure may use modular pre-bent off-the-shelf knee stabilizers, and self-contouring structural fabric panels to form around the knee shape, with no fabrication required.
18. The prior Martin disclosure requires a prosthetist to fit it to an amputee.
   18.1. This disclosure's fitting process is simple enough to be assembled by an amputee for themself.
19. The prior Martin disclosure supporting structure is affixed and anchored to the Struts.
   19.1. This disclosure may be removable from the structure and simply suspends or hangs from them, via connectors mechanisms, in a preferred embodiment.
20. The prior Martin disclosure BK uses a wide carbon fiber panel or two anchored anterior carbon fiber or aluminum struts to support the limb, which are typically custom molded to the user, with two to three posterior floating struts which can be pulled toward each other for tightening around the posterior aspect of the limb.
   20.1. This disclosure may use one anterior anchored support structure, and two anchored posterior support structures-none of which can be tightened toward each other, and none of which are custom molded to the user.
21. The prior Martin disclosure requires the use of a gel liner over the residual limb to fit within the socket, in order to contain and control the limb tissue.
   21.1. This disclosure may not require a gel liner, as it may leverage an encapsulated velcro-compatible soft sock within the structural fabrics to fully contain and support the limb tissue, in coordination with the containment straps and composite structural panel assembly.
22. The prior Martin disclosure uses pre-defined sub-components to form together around the general limb shape in a finite number of predetermined configuration options.
22.1. This disclosure uses sub-components which may be formed together in an infinite number of placements, configurations, and positions to fully customize the socket shape to the specific limb shape, versus a general limb shape.

In each of the above prior art, conventional casting/impression, modification, and iterative fitting processes are required to generate a custom contoured socket shape to fit around at least various areas of the limb, in which at least a portion of the user's weight is bore through the custom contoured non-compliant materials.

### SUMMARY OF THE INVENTION

The present invention relates generally to an apparatus, system, and method of fitting a prosthesis for amputees. More particularly, the present invention is a new and improved prosthetic socket apparatus, system, and method utilizing conforming structural fabrics to provide increased comfort to the user, and which does not require a highly-trained prosthetist or conventional fabrication machinery to perform the prosthetic fitting.

In this respect, before explaining at least one embodiment of the invention in detail, it is to be understood that the invention is not limited in this application to the details of construction and to the arrangement of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods and systems for carrying out the several purposes of the present invention. It is important, therefore that the claims be regarded as including such equivalent constructions insofar as they do not depart from the spirit and scope of the present invention.

Accordingly, titles, headings, chapters name, classifications and overall segmentation of the application in general should not be construed as limiting. Such are provided for overall readability and not necessarily as literally defining text or material associated therewith.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

It is therefore an object of the present invention to provide a new and improved method of fitting a prosthetic socket.

It is an object of the present invention to make the prosthetic fitting to be accomplished in a single appointment.

It is an object of the present invention to accomplish a prosthetic fitting in less than an hour from start to ambulation.

It is an object of the present invention to be able to fit a prosthetic socket without requiring specialized fabrication equipment commonly used in making prosthetics.

It is an object of the present invention to be able to fit a prosthetic socket with only requiring simple hand tools such as, but not necessarily limited to, scissors and Allen wrenches.

It is an object of the present invention to eliminate the requirement of a highly trained Prosthetist to perform the prosthetic fitting.

It is an object of the present invention to enable a layman or amputee to fit their own prosthetic socket, thanks to the intuitive simplicity of the fitting method and process.

It is an object of the present invention to use a standard set of sub-components to be able to fit many types and sizes of residual limbs.

It is an object of the present invention to use a standard set of sub-components which can be assembled in a way as to fully customize the fit to the user's unique limb shape.

It is an object of the present invention to provide a prosthetic socket shape that fully conforms to the amputee's limb shape in real-time.

It is an object of the present invention to provide exceptional comfort for the amputee.

It is an object of the present invention to reduce discomfort issues typically experienced in conventional prosthetic such as but not limited to high point-pressures, rubbing, chaffing, and the like.

It is an object of the present invention to provide un-impinging and unrestricted range of motion of the limb while wearing the prosthesis.

It is an object of the present invention to provide excellent heat dissipation, thereby making the prosthetic socket feel cool and breathable.

It is an object of the present invention to provide a prosthetic socket that is user-adjustable to accommodate changes in the residual limb.

It is an object of the present invention to provide a prosthetic socket that promotes limb muscle usage, and therefore provides an increase in limb tissue circulation and muscle mass growth.

It is an object of the present invention to provide a prosthetic that provides increased prosthetic limb proprioception for the wearer.

It is an object of the present invention to provide a prosthetic socket that is user repairable, and in most parts of the world, with simplicity and commonly available hand tools.

It is an object of the present invention to provide a prosthetic socket that is machine washable, similar to a garment.

It is an object of the present invention to provide a prosthetic socket that is ultra-durable and long-lasting.

It is an object of the present invention to utilize structural fabrics to maximize the strength and integrity of the exoskeleton structure, via tensioning the structural fabrics around them and prevent bowing of the structure.

It is an object of the present invention to provide a prosthetic socket that is low cost to the amputee, and which has a low cost of manufacture.

It is an object of the present invention to provide a prosthetic socket that is visually appealing to the psychology of an amputee.

Another object of the present invention is to provide a new and improved system which provides some of the advantages of the prior art, while simultaneously overcoming some of the disadvantages normally associated therewith.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference would be had to the accompanying drawings and descriptive matter in which there are illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed pictorial illustrations, graphs, drawings, and appendices.

FIG. 1 generally illustrates a preferred embodiment of the invention, as a prosthetic socket system for above knee (AK) and below knee (BK) levels, with the below knee socket shown from an anterior lateral perspective view, and the above knee socket shown from the posterior lateral view.

FIG. 2A illustrates an embodiment of various structural fabric sub-components which may be used in various combinations to form a prosthetic socket or other such interface system, preferably for an AK prosthesis.

FIG. 2B illustrates an embodiment of various structural fabric sub-components which may be used in various combinations to form a prosthetic socket or other such interface system, preferably for an BK prosthesis.

FIG. 3A generally illustrates configuration steps for assembling various sub-components around a BK residual limb to form a prosthetic socket.

FIG. 3B generally illustrates configuration steps for assembling various sub-components around an AK residual limb to form a prosthetic socket.

FIG. 4 generally illustrates an embodiment of a layer of structural fabrics about a BK limb, presented as a velcro-compatible sock.

FIG. 5 generally illustrates various structural fabric panel sub-components which may be assembled and combined together in various combinations to form to the specific shape of a residual limb, and even the full residual limb, shown for a BK and AK socket level.

FIG. 6A generally illustrates an embodiment of a containment strap system used to tighten around a limb.

FIG. 6B generally illustrates an embodiment of a containment strap system used to tighten around a limb.

FIG. 6C generally illustrates an embodiment of a containment strap system suing to tighten around a limb.

FIG. 7A generally illustrates posterior lateral views of a below knee socket using structural fabrics to form a brim shape.

FIG. 7B-1 generally illustrates a medial view of an AK socket using structural fabrics to form a brim shape.

FIG. 7B-2 generally illustrates a medial-posterior view of an AK socket using structural fabrics to form a brim shape.

FIG. 8 generally illustrates an embodiment of an exoskeleton support structures for below knee (A) and above knee (B) sockets, and used to support a structural fabric flexible inner socket assembly.

FIG. 9 generally illustrates various embodiments of a modular foundation base of an exoskeleton support structure using two, three, or four extensions for support of a structural fabric flexible inner socket.

FIG. 10 generally illustrates an overlay of various sizes, with various bend curvatures, of extensions mounted to the base of an exoskeleton structure used to support a structural fabric flexible inner socket assembly, for both below knee (A) and above knee (B) configurations.

FIG. 11 is a posterior views of a below knee exoskeleton structure used to support a structural fabric flexible inner socket assembly, whereas (pre-bent, though the bend is not shown) knee stabilizers' angles may be adjusted using a non-backdrivable gear mechanism.

FIG. 12 generally illustrates proximal views of a below knee exoskeleton support structure used to support a structural fabric flexible inner socket assembly, whereas base extendor extension angles may be adjusted using a non-backdrivable gear mechanism.

FIG. 13A generally illustrates an exoskeleton structure for extending proximal to a below knee socket, which may be used for suspension, control, and/or stability.

FIG. 13B generally illustrates an another embodiment of an exoskeleton structure for extending proximal to a below knee socket, which may be used for suspension, control, and/or stability.

FIG. 14 generally illustrates an exoskeleton structure for extending proximal to a below knee socket, which may be used for suspension, control, stability, and/or offloading weight from the below knee limb.

FIG. 15 generally illustrates a lock mechanism and liner used for suspension of a limb within a socket, but which may not be directly attached to the socket.

FIG. 16 generally illustrates the relationship of a limb, liner, socket, and lock mechanism used for suspension of a limb within a socket, but which may not be directly attached to the socket.

FIG. 17 generally illustrates an adjustor mechanism which may be used to tighten structural fabric materials around a limb, shown from both the front and perspective views.

FIG. 18A generally illustrates a lateral view and a perspective view of a modified below knee conventional rigid socket with lowered trim lines and replaced with conforming and adjustable modular structural fabric materials, including a fabric-brim.

FIG. 18B generally illustrates an anterior-medial and anterior-lateral perspective views of a modified above knee conventional rigid socket with lowered trim lines and replaced with conforming and adjustable modular structural fabric materials, including a fabric-brim.

FIG. 19A generally illustrates an anterior-lateral and posterior-lateral perspective views of a modified below knee conventional rigid socket with lowered trim lines and replaced with conforming and adjustable modular structural fabric materials, including a fabric-brim, and whose conventional rigid socket trim lines having been lowered greater than example FIG. 18A.

FIG. 19B generally illustrates an lateral view and posterior-medial perspective view of a modified above knee conventional rigid socket with lowered trim lines and replaced with conforming and adjustable modular structural fabric materials, including a fabric-brim, and whose conventional rigid socket trim lines having been lowered greater than example FIG. 18B.

### DETAILED DESCRIPTION OF THE PREFERRED EMBODIMENTS

In a preferred embodiment, the current invention may include the following although it is contemplated that combinations, modifications, or various configurations may be utilized to provide similar effectiveness, and so forth as generally referred to in the application and illustrations described below. Therefore, nothing contained herein shall be considered limiting. It is contemplated that similar means as the below may be used to establish a physical man/machine interface for use-cases other than for prosthetic sockets, including orthotics, exoskeletons, backpacks, shoes, sports equipment, casts for broken limbs, or any other apparatus which is used in contact with the human body, whose contact is for at least partially supporting a portion of the body, and whose contouring is specific to the user's body shape.

### Structural Fabric Flexible Inner Socket

In the enclosed descriptions, the term structural fabrics generally refers to the use of materials with the following characteristics:

In a preferred embodiment, they may be woven, though they may also be non-woven.

In a preferred embodiment, their materials may be conforming.

In a preferred embodiment, they may be able to be connected together by various methods, preferably using a hook and loop velcro-like compatibility, though other common connection methods such as but not limited to sewing, bonding, buckles, and the like may also be used.

In a preferred embodiment, they may be considered to be a fabric, though may also not be a fabric.

In a preferred embodiment, they may have sufficient tensile strength for their intended purpose.

In a preferred embodiment, they may be non-elastic in nature for use-case where non-elasticity is important, or may be elastic in nature for use-case where elasticity is important.

In a preferred embodiment, their individual nature alone may not be suitable for a biomechanical support of a limb within a socket or other such interface, but their combination as described herein may.

In a preferred embodiment, they may be uncommon materials or have uncommon use as for supporting the limb in a prosthetic socket or similar interfaces described herein.

In a preferred embodiment, they may be too conforming to enable the support of a limb without the integration of an exoskeleton structure, or the like.

In a preferred embodiment, they may be machine washable.

In a preferred embodiment, they may be breathable materials, allowing air to pass through.

In a preferred embodiment, they may allow water to pass through, as they may not be watertight.

In a preferred embodiment, they may not individually be able to adequately contour to or support a limb, without their combination or assembly, such as is described herein.

In a preferred embodiment, they may be died to various colors.

In a preferred embodiment, their size or length may be adjustable, pre or post assembly.

In a preferred embodiment, their size or length may be modifiable with scissors.

In a preferred embodiment, their shape may, at least partially, be dictated by the forces imposed upon them, versus being pre-formed to certain established shapes as conventional sockets are.

In a preferred embodiment, they may be conforming enough to crumple up in hand, yet sturdy enough to support the limb.

In a preferred embodiment, the combination of various structural fabric materials, elasticity, connectivity, and tensile strengths may assemble into a socket, whereas any one of them alone would be insufficient to accomplish the same.

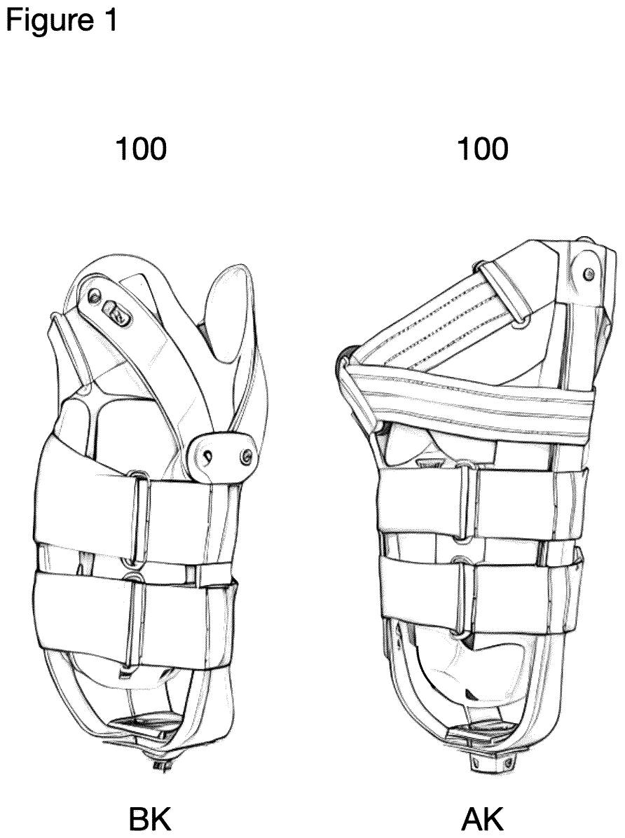

As is illustrated in FIG. 1, the invention 100 is a prosthetic socket for an amputee, or other such man/machine interface, which may utilize structural fabrics to encompass, control, support, contain, connect to, and/or establish a limb. In such an example, both a below knee and above knee socket assembly are illustrated, although it is contemplated that similar methods may be used for other levels of amputation or other such use-cases such as but not limited to above elbow or below elbow amputation levels and sockets thereof, using generally similar means. In the illustration, the below knee structural fabric inner socket is shown with its exoskeleton structure attached, while the above knee structural fabric inner socket is shown without its exoskeleton attached. Through the combinations of various structural fabric sub-components, a structural fabric flexible inner socket may be formed, and which may be specifically customized around a specific residual limb shape.

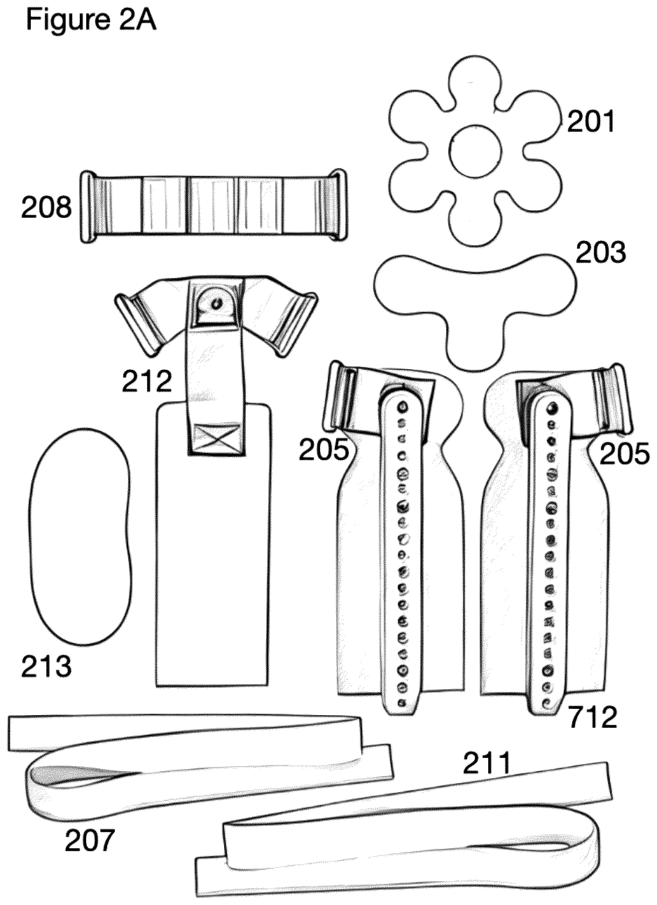

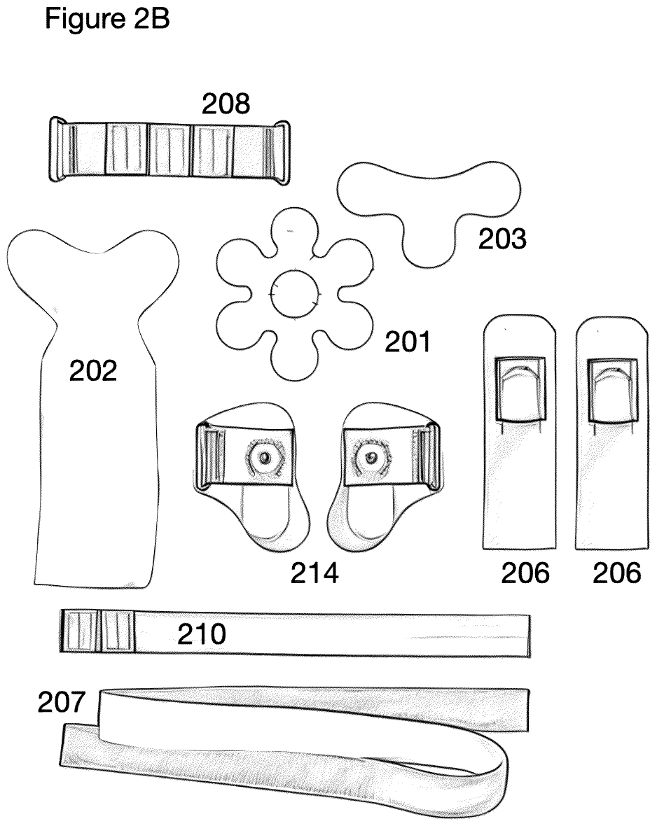

FIG. 2 illustrates embodiments of various structural fabric sub-components which may be use to form a prosthetic socket or other such interface system. In the illustrations, the various structural fabrics, combinations, and shapes may be made of various types of materials, including but not limited to: woven materials, non-woven materials, velcro compatible hook and/or loop materials, interlocking fabric connectivity materials, various thicknesses of materials, various stiffnesses of materials, and the like. In a preferred embodiment, any such velcro-compatible materials may use near-permanent connection types of velro-compatibility, such that the hook and loop adhesion can be strong enough to support a user through the use-case of ambulating on such a device. Such materials may be connected together in layers or combinations by: sewn methods, laminated or bonded methods, snaps, hooks, bonding agents, friction, adhered, and the like. The term adhered, as used in this description may include various bonding methods, which may encompass methods of glueing or otherwise bonding, or may refer to velcro-like-compatible hook sticking to velcro-like-compatible loop. None of which should be considered limiting, and the descriptions used are for general understanding purposes only, though the descriptions used are a practical and viably workable solution to accomplish a successful prosthetic socket fitting.

In a preferred embodiment, certain materials may be velcro-like velcro-compatible stretchy materials, certain materials may be an bonded combination of non-stretchy velcro-compatible hook-fabric-loop materials, certain materials may be webbing, certain materials may be velcro-like hook, certain materials may be velcro-like loop, certain materials may be high friction materials against other certain materials, certain materials may be purposefully low friction materials against other certain materials, certain materials may include leather, certain materials may include hide with or without fur, and certain materials may include foams, amongst others. It is understood that various other forms and types of materials may be used to. Altogether, the various structural fabric materials may be used in a combination to provide the desired properties, such as stretchiness, non-stretchiness, bendability, elongation properties, durometer characteristics, cushioning, strength and durability, and other characteristics.

For illustrative purposes the following sub-components may include:
201: Distal umbrella, for placement over the end of a residual limb. Such sub-component may be made from non-stretch bonded near-permanent hook-fabric-loop materials to provide durability, flexibility yet enough strength and resistance to bending to provide form, and to provide simple connectivity to other structural fabric layers.
202: Anterior panel, for placement over the front of a below knee residual limb. Such sub-component may be made from non-stretch bonded near-permanent hook-fabric-loop materials to provide durability, flexibility yet enough strength and resistance to bending to provide form, and to provide simple connectivity to other structural fabric layers. The distal end of such a panel may connect to 201.
203: T-panel, a configuration option for placement in connection with the extensions from the distal umbrella 201 to create an assembly of a bowl-shape. Or may be used in other areas such as to create contouring at the brim level of a socket for example. Such sub-component may be made from non-stretch bonded near-permanent hook-fabric-loop materials to provide durability, flexibility yet enough strength and resistance to bending to provide form, and to provide simple connectivity to other structural fabric layers.
205: AK Medial Panels, for placement over the medial aspect of the AK limb, and may or may not be connected together to create a unified panel, for an above knee socket, and to provide a connection point between an exoskeleton structure and a structural fabric inner socket. Such sub-component may be made from a non-stretch fabric layer(s) sewn or connected to near-permanent hook-fabric-loop materials, for example, to provide simple connectivity to other structural fabric layers. The proximal end of such AK Medial Panels may be incorporated as part of a brim assembly. The distal end of such a panel may connect to 201.
206: BK Posterior Panel/Sleeves, for placement over the posterior aspect of the BK limb, and may or may not be connected together to create a unified panel, for a below knee socket, and to provide a connection point between an exoskeleton structure and a structural fabric inner socket. Such sub-component may be made from a non-stretch fabric layer(s) sewn or connected to near-permanent hook-fabric-loop materials, for example, to provide simple connectivity to other structural fabric layers. The proximal end of such Posterior Panels may be incorporated as part of a brim assembly. The distal end of such a panel may connect to 201.
207: Posterior containment strap section for below knee socket, or medial containment strap for above knee socket. Such sub-component may be made of non-stretch webbing or fabric and may include connectivity means such as velcro-like materials to adhere to other structural fabric layers. Such strap may generally work in coordination with 208 to provide circumferential containment about a limb.
208: Anterior containment strap section for below knee socket, or lateral containment strap for above knee socket. Such sub-component may be made of non-stretch webbing or fabric and may include connectivity means such as velcro-like materials to adhere to other structural fabric layers. Such strap may generally work in coordination with 207 to provide circumferential containment about a limb.

209: Buckle loop, used as a connection point for straps, such as 207, 208, 210, or others, to route through, and used to control the direction of webbing and resultant forces about the limb.
210: V-strap for below knee socket posterior brim. Such sub-component may be made of non-stretch webbing or fabric and may include connectivity means such as velcro-like materials to adhere to other structural fabric layers. Such strap may generally connect to the buckle loops on 214, route through opposing side buckle loops on 208, and back through adjustor mechanism 604.
211: AK Brim Strap for above knee socket brim. Such sub-component may be made of non-stretch webbing or fabric and may include connectivity means such as velcro-like materials to adhere to other structural fabric layers. Such strap may generally connect to the buckle loops on 205 and 212, and back through adjustor mechanism 604.
212: Lateral sleeve and brim anchor point for above knee socket. Used to provide a connection point between an exoskeleton structure and a structural fabric inner socket, and effectively to hang the lateral side of the structural fabric inner socket from, which may include the brim straps. Such sub-component may be made from a combination of various non-stretch fabric layer(s) sewn or connected to near-permanent hook-fabric-loop materials, for example, to provide simple connectivity to other structural fabric layers. The distal end of such a panel may connect to 201.
213: Modular pad, which may be positioned generally longitudinally along the limb, and which the containment straps may press against in order to put additional pressure into the soft tissue of the limb, and which may be used to assist in providing limb rotational control within the socket. These may be made of foam, fabrics, plastics, silicons, or any number of other materials which may provide conformity to the limb shape, or which may have enough rigidity to cause the limb shape to conform to its shape. Such a sub-component may have velcro-like attachment means for modularity adhering to other structural fabric materials. Similarly, they may be used at the brim-level for an above knee socket, under the brim-level containment straps, to provide extra passing or cushioning.
214: Knee Stabilizer panels for below knee socket, which may be adhered to other structural fabric panel sections, such as the anterior panel 202, or frame structure 801, and which may be positioned generally along the sides of the anatomical knee areas. Used to provide a connection point between an exoskeleton structure 801 and a structural fabric inner socket, and effectively to hang the structural fabric inner socket from along the anterior medial and anterior lateral aspects, which may include the brim straps. Such sub-component may be made from a combination of various non-stretch fabric layer(s) sewn or connected to near-permanent hook-fabric-loop materials, for example, to provide simple connectivity to other structural fabric layers. Likewise, such materials and shape may be used to provide broad conformity around the anatomical knee shape, through using materials that have flexibility yet enough strength and resistance to bending to provide form, and to provide simple connectivity to other structural fabric layers.

It is understood that stretchy fabrics may also be used where non-stretch fabrics are noted. However, it has been found that stretch characteristics may cause an unstable sensation, as there may be a loss of one-to-one control of the prosthetics biomechanics, as direct connection forces may be lost in the material stretch properties.

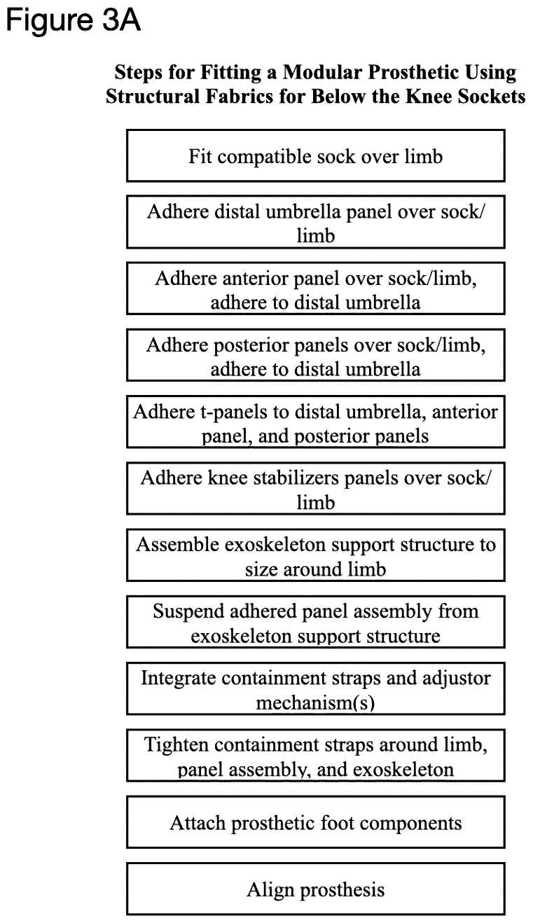

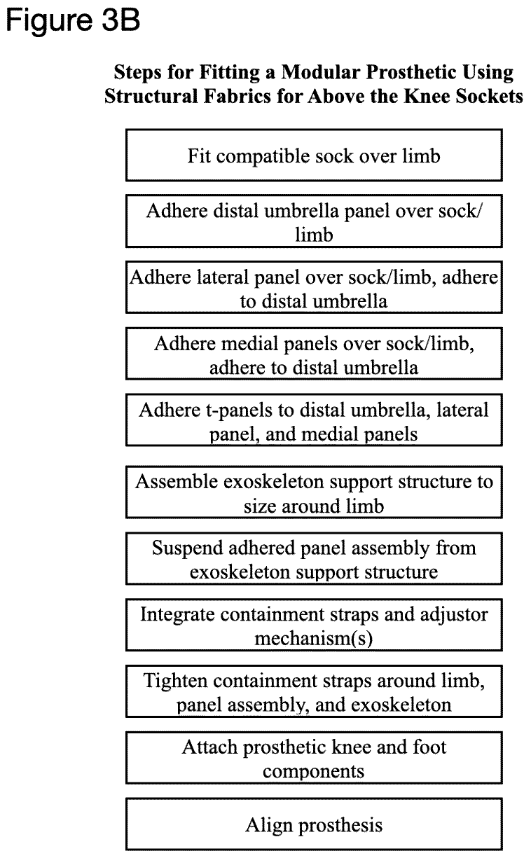

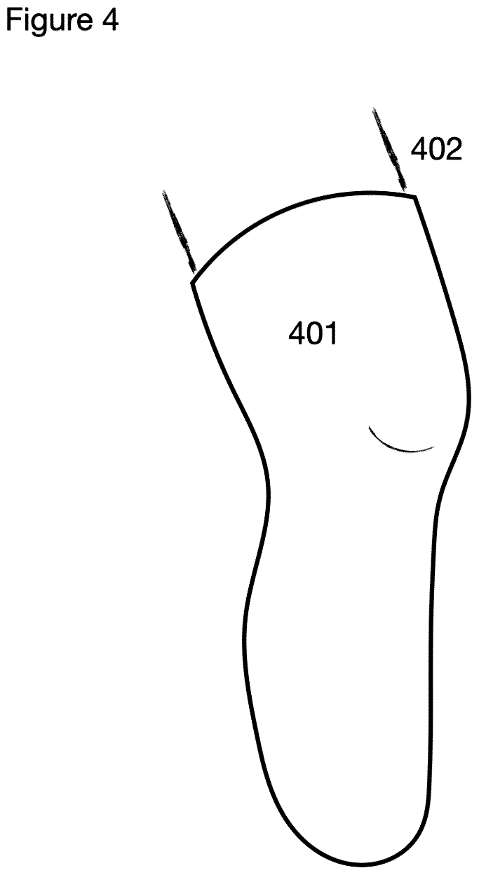

FIG. 3A generally illustrates a configuration option for the steps of assembling various sub-components around a below knee residual limb to form a structural fabric flexible inner socket, to connect to an exoskeleton structure, and generally to complete the fitting of a socket for an amputee.

FIG. 3B generally illustrates a configuration option for the steps of assembling various sub-components around an above knee residual limb to form a structural fabric flexible inner socket, to connect to an exoskeleton structure, and generally to complete the fitting of a socket for an amputee.

In both FIGS. 3A and 3B, the general order of assembly may be used, or alternative orders are contemplated, and such description should not be considered limiting. In general, there may be various layers of structural fabric materials which may be used in combination to establish the dynamic characteristics of a prosthetic socket for example.

In one embodiment, the assembly steps may generally include various combinations of a foundation sock, structural fabric sections and panels, exoskeleton attachment, and containment straps, though such combination should not be considered limiting. To accommodate for varied limb sizes and dimensions, the structural fabric sub-components may be customized to the user by placing them in the best anatomical positioning with respect to that individual limb requirements. The stretchiness of the sock layer, along with the customized positioning of the other non-stretchy structural fabrics that attach to the sock, in total creates a uniquely shaped socket, with unique characteristics.

A velcro-compatible loop sock (401) may be donned over the residual limb (402), which may provide slight compression of the limb tissue, containment of the limb tissue, and contouring of the limb tissue. Such sock is not required for such a fitting, but may be advantageous for simplifying the the other structural fabric panels thereto. Placing a distal umbrella over the end of the limb (201) and sock 401, may provide contouring around the end of the limb, as it's shape allows it to contour 3-dimensionally to a ‘bowl’ shape. The distal umbrella (201) may adhere to the velcro-compatible sock (401) by various means, such as it's velcro-compatibility. The distal umbrella (201) may be made of hook-fabric-loop bonded together, allowing both sides to be velcro-compatible, thereby allowing other sub-components to be adhered to the top of it. It is clearly understood that it may also be fabric-hook, loop-hook, or other such combinations.

In a similar way, an anterior panel (202) may be placed over and adhered to the sock (401) in an anatomical position to contour over the anterior aspect of the BK residual limb for example. This component may allow for a dispersion of forces around the sensitive anterior bony structures of the limb, yet provide full conforming contouring to the limb shape with soft-compliant means.

In a preferred embodiment, two t-panels (203) may be used to adhere to various of the extensions of the distal umbrella (201), which may create a larger portion of a bowl shape. More particularly, they may also connect between the anterior panel and posterior panels of a BK socket, and lateral panel and medial panels of an AK socket, and also to the distal umbrella 201, thereby creating a general bowl shape to match the shape of the end of the limb. The specific contouring of the various t-panel shapes is such that as they connect to the distal umbrella (201) and wrap around the distal end of the limb shape circumferentially, they create a general bowl shape in combination with each other and with a distal umbrella.

Next, another set of panel may be placed over the posterior aspect of below knee the limb (206), whose top may be purposefully placed to contour into and with the height of the brim area of the socket. The proximal end of such a panel may be made of one or more pieces, which together or separate may generally form a shape of posterior brim. This methodology allows for multiple directions of contouring around a 3-dimensional limb shape, with using otherwise flat sections of structural fabrics bonded together.

Similarly, sleeve panels (206) may be placed in position across the posterior medial and posterior lateral aspect of the limb, with their tops at or near the desired brim height of the socket. Such sleeve panels may be connected together or not, together forming the general posterior aspect of the socket. Their top may not be connected together, allowing them to better contour to the limb shape. In such an example, the top brim may be configured from soft webbing containment straps alone, and not by a specifically contoured panel.

### Systematic Fitting Steps

In order to provide additional clarity and a more systematic fitting steps of a preferred embodiment, the following description is provided for BK and AK sockets.

### General Steps for Fitting a Modular Prosthetic Using Structural Fabrics for Below the Knee Sockets

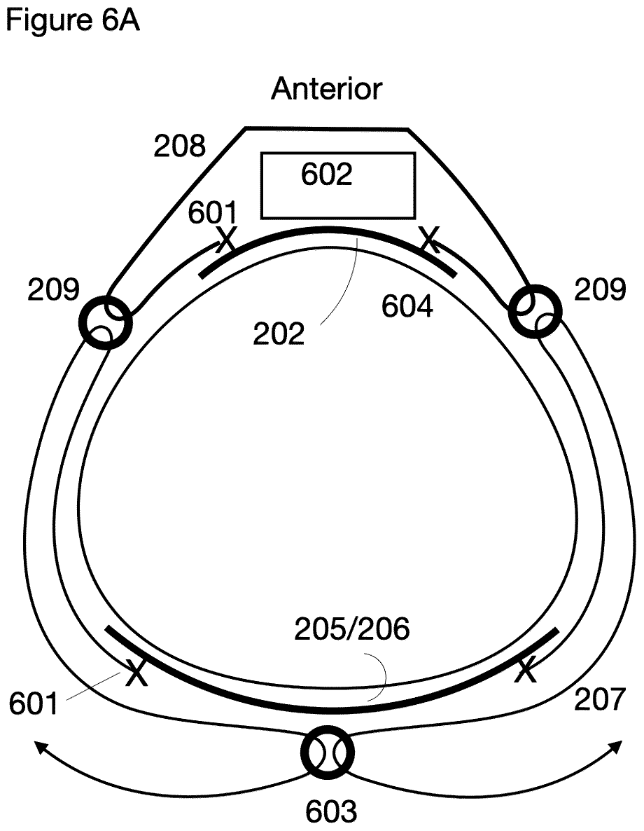

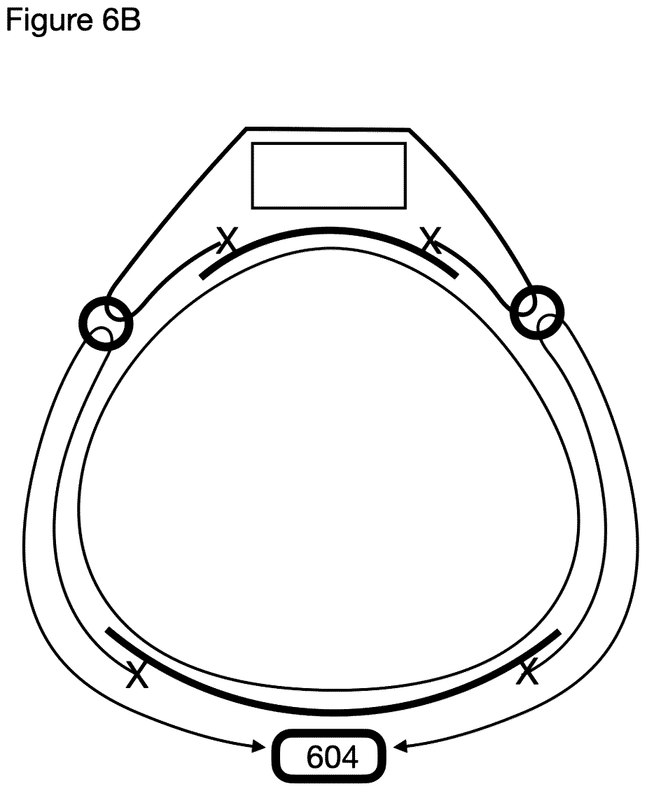

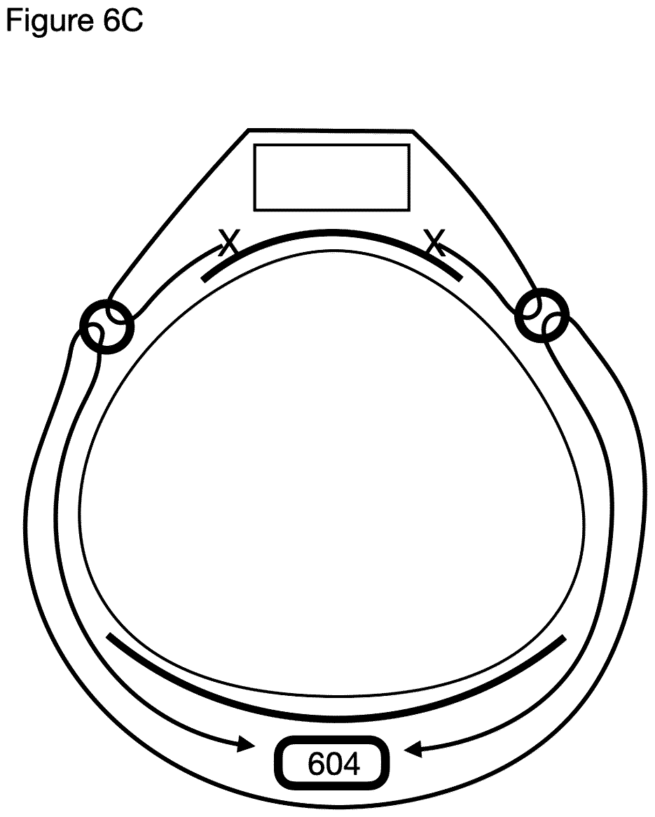

1. Fit the velcro-compatible sock 401 over the limb, pulled tight.
2. Place and adhere the distal umbrella 201 over distal end of the limb and sock 401. Pull each of the arms of the distal umbrella snug around the end of the limb, with opposite side arms being pulled together to contour its shape to the limb shape.
3. Place and adhere the anterior panel 202 over the limb and sock 401, lining up the top with the mid-patellar tendon (below the kneecap).
4. Cut anterior panel 202 to length, so that it overlaps and adheres to the distal umbrella 201. If there is puckering, snip a long triangle out of the bottom center of the anterior panel 202, so that it contours to the limb contouring.
5. Place and adhere the posterior panels 206 onto the limb and sock 401, with their proximal end lined up with the desired brim height, and spread apart at about 2/3 of the limb width.
6. Trim the posterior panels 206 to length, so that they overlap and adhere to the posterior arms of the distal umbrella 201.
7. Place a t-panel 203 to connect its arms between the anterior panel 202 and posterior panel 206 on each side of the limb, pulled tight. The bottom of the t-panel 203 should connect to the distal umbrella arms 201, thus creating a tight-fitting bowl-like shape contoured to the end of the limb.
8. Ensure that the base extenders 806 are sized correctly, so that there is enough room between the spine 808 and sbones 802 fit around the limb with contouring, but not too tight in the anterior to posterior dimension. Adjust the base extenders 806 to foundation 805 anchor angle as necessary to ensure the base extenders are set wide enough apart for the limb size. Swing the base extenders 806 angle wider to accommodate a larger limb, and to line up with the preferred sbones 802 sleeve/panel placement.
9. With the top base of the foundation 805 roughly 25 mm to 50 mm below the end of the limb (if limb length permits), adjust the sbones 802 length and width so that their top is at the top of the posterior panel 206 sleeve height. Slide the sbones 802 into the connector sleeves on the posterior panels 206.
10. With the top base of the foundation 805 roughly 25 mm to 50 mm below the end of the limb (if limb length permits), adjust the spine 808 length so that the top of the knee stabilizers 801 are at the middle sides of the anatomical knee joint.
11. Connect the knee stabilizers 801 onto the knee stabilizer panels 214. Place a plastic bag over the panels, and position the knee stabilizers 801 and their connected panels 214 around the anatomical knee in the appropriate position.
12. Slide the plastic bags out slowly, thereby adhering each knee stabilizer panel 214 to the anterior panel 202 and sock 401, one at a time. The fabric inner socket is now assembled and connected to the exoskeleton.
13. The proximal containment strap assembly uses the brim straps 207 anchored into the buckles of the knee stabilizer panels 214, then wraps around the posterior aspect of the limb, and through the buckles on the opposing side's anterior containment strap 208, then reverses toward the back of the limb, and through an adjustor mechanism 604.
14. Attach a second containment strap assembly by placing an anterior segment of the containment strap 208 around the front of the limb, just below the height of the proximal brim-level containment strap, and on the outside of the spine 808. Then at the same height as the anterior containment strap 208, route the posterior containment strap 207 around the limb, through the buckles on the ends of the anterior containment strap 208, and back through the adjustor mechanism 604, as illustrated in FIG. 6C for example.
15. As limb length permits, leaving a small gap in between the individual containment straps, add additional containment straps down the limb length. The bottom containment strap should not overlap the proximal end of the distal umbrella assembly 201/202/203 by more than 10-15 mm.
16. Align the socket using conventional socket alignment methods.

### General Steps for Fitting a Modular Prosthetic Using Structural Fabrics for Above the Knee Sockets

1. Ensure that the base extenders 806 are sized correctly, so that there is enough room between the spine 803 and sbones 804 to fit the limb with contouring, but not too tight medial to lateral. Adjust the base extenders 806 to foundation anchor 805 angle width as necessary. Swing the base extenders angle wider to accommodate a larger limb, and to line up with the preferred medial panel 206 placement.
2. Connect the medial panels 206 to the sbones 804.
3. With the top of the foundation 805 roughly 25 mm to 50 mm below the end of the limb (if limb length permits), adjust the sbones 804 length so that the top of the connected medial panels 206 lines up with the top of the desired brim height.
4. With the top of the foundation roughly 25 mm to 50 mm below the end of the limb (if limb length permits), adjust the spine 803 length so that the top of the spine 803 is roughly 25 mm above the trochanter height.
5. Place the anterior medial sbones panel 206 centered with the adductor longus tendon at the desired brim height.
6. Place the posterior medial sbones panel 206 lining up the top of the panel directly under the ischial tuberosity at the desired brim height. The posterior medial sbones panel 206 may be up to about 25 mm higher than the anterior medial sbones panel 206.
7. Fit the velcro-compatible sock 401 over the limb, pulled tight.
8. Place the distal umbrella 201 over distal end of the limb and sock 401. Pull each of the arms of the distal umbrella snug around the end of the limb, with opposite side arms being pulled together to contour its shape to the limb shape.
9. Trim the medial panels 206 to length, so that their length overlaps and connects to the distal umbrella 201.
10. Connect the lateral panel 212 to the top of the spine 803.
11. Cut the lateral panel 212 to length, so that it overlaps and adheres to the distal umbrella 201. If there is puckering, snip a long triangle out of the bottom center of the lateral panel 212, so that it contours to the limb contouring.
12. Place a t-panel 203 to connect its arms between the lateral panel 212 and medial panels 206 on the front and back sides of the limb, pulled tight. The bottom of the t-panel 203 should connect to the distal umbrella arms 201, thus creating a tight-fitting bowl-like shape contoured to the end of the limb.
13. The proximal containment strap assembly uses the brim straps 210, which may route around the outside of the lateral aspect of the limb and spine 803 toward the medial aspect, then may route through both of the buckles of the medial panels 206, then may route through the buckles on the lateral panel 212, then may loop back toward itself and modularly connect overlapping the same brim straps 210 between the medial panels 206 and the lateral panel 212. It may also leverage the use of adjustor mechanisms 604, or the like, to accomplish the adjustability.
14. Attach a second containment strap assembly by placing a lateral segment of the containment strap 208 around the lateral aspect of the spine 803, just below the height of the proximal brim-level containment strap. Then at the same height as the lateral containment strap 208, route the medial containment strap 207 around the limb, through the buckles on the ends of the lateral containment strap 208, and back through the adjustor mechanism 604, as illustrated in FIG. 6C, for example.
15. As limb length permits, leaving a small gap in between the individual containment straps, add additional containment straps down the limb length. The bottom containment strap should not overlap the proximal end of the distal umbrella assembly 201/202/203 by more than 10-15 mm.
16. Align the socket using conventional socket alignment methods.

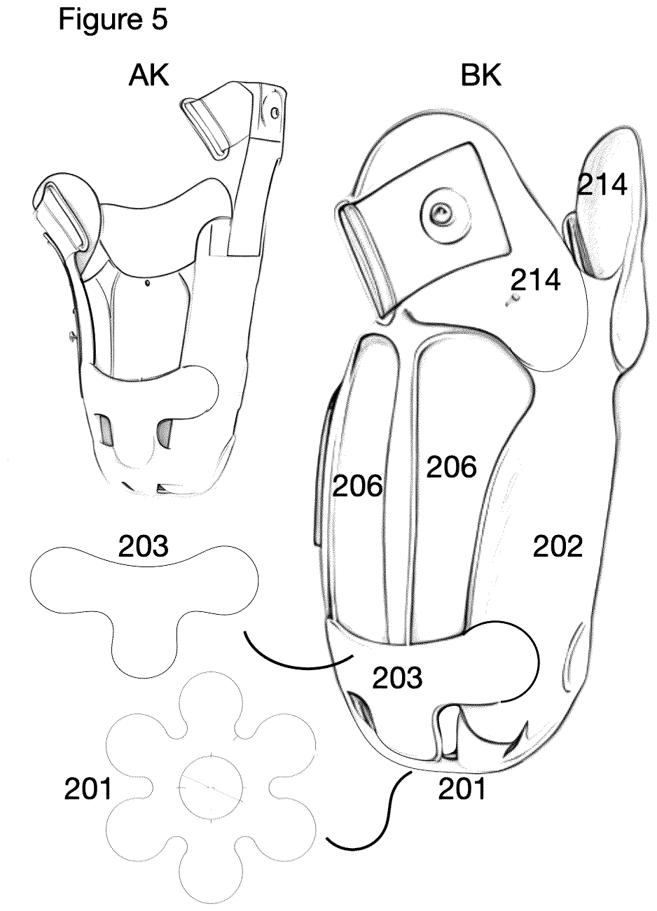

As is illustrated in FIG. 5, shown for a below knee and above knee socket example, at the distal end of the limb, connecting a distal umbrella (201), with t-panels (203) to connect the sides of each to an anterior panel 202 (BK) and posterior panels (206) together completes a circumferential bowl shape around the distal end of the limb. It is contemplated that the same concept can be configured for an above knee socket, using its distal umbrella, t-panels, medial panels, and lateral panel to create the similar bowl shape to contour to the end of a limb. As illustrated in FIG. 5, the more proximal assembly of the anterior panels (202), sleeve panels (206), distal umbrella (201), and t-panels (203) may be used to form a span up the front and back of the BK (below the knee) limb, all the way around a distal bowl shape of the BK limb. These structural fabric elements may, or may not, be attached or adhered over the top of the velcro-compatible sock (401). It is an object of the present invention to leverage the distal end assembly, including 201 and 203, and along with 202 and 206 and 214, to contain and control the limb tissue within the socket, and prevent the limb tissue from splaying outward upon weight bearing. Likewise, they may be used to provide additional biomechanical limb control and dynamics within the socket. For the AK example, the lateral panel and medial panel assemblies may extend upward toward the proximal end of the socket's brim level.

The additional layers which may be added, such as sleeve sections (206) and knee stabilizer panels 214 may be used as an anchoring method for an external structure, and may be used to support the limb. Since it is a preference for the structural fabric flexible inner socket to be removable from the external frame structure (herein referred to an an exoskeleton), sleeves or other connector means may be one of various options to use as a removable connection point. This may allow the various sections of the exoskeleton to slide into the sleeves for example, which then supports the limb via the structural fabric inner socket, in a removable way.

Various connector sections may be used around the structural fabric inner socket assembly, which may include, for the BK socket configuration, two posterior sleeves (206), and a connector means on each side of the knee stabilizer panels (214). Each of these methods may have a portion of the exoskeleton structure connect to it, to support a portion of the limb.

Once all sub-components are assembled together, for example, the connectors for the proximal end of the BK exoskeleton structure which are positioned along the sides the anatomical knee may overlap and adhere to the anterior panel and knee stabilizer panel area. Therefore, the exoskeleton structure which may be connected to such panels will allow 214, 202, and its ultimate connection down to 201 all to be suspended from such connectors. Likewise, the sleeve panels 206 may be connected to 201 and may support the limb's weight though the structural fabric inner socket. In total, the user's body weight and limb forces may be supported through the structural fabric inner socket, which is suspended from the exoskeleton.

For the AK (above knee) socket, there may be two medial panels 205 and one lateral panel 212 to support the limb instead. The brim straps may be suspended from the lateral panel's proximal webbing area, as well as from the medial panel assembly.

While the elastic sock 401 may be advantageous as a common structural fabric component to adhere the other structural fabrics to, it is understood that such elastic sock 401 may be left out or removed from such a combination, allowing the anterior panel, posterior panel, distal umbrella, containment straps, and other such sub-components to remain assembled around the limb, but without utilizing a commonly connected elastic sock 401.

Once assembly and sizing adjustments are complete, containment straps (207, 208) may be placed around the limb. It is an object of the present invention to leverage the containment straps for multiple purposes. Containment straps may be generally used to contain and control the limb tissue within the socket, and prevent the limb tissue from splaying outward upon weight bearing. Likewise, they may be used to provide additional biomechanical limb control and dynamics within the socket, such as compressing the shape of various panels inward toward the limb, and hence contouring the panels to the limb shape in real-time. Furthermore, they may be used to provide increased structural integrity of the exoskeleton and overall socket structure, as they may effectively tie the full set of sub-components together circumferentially and prevent them from bowing outward under load, including supporting the exoskeleton from bowing.

Various combinations and methods may be used to circumferentially contain the limb with containments straps. Those described in this disclosure should not be considered limiting, but rather are used for illustrative purposes. However, those described are an effective and unique method of containing the limb tissue within a prosthetic socket.

As is illustrated in FIG. 6A and FIG. 6B, it has been discovered that the described embodiment is an excellent method of accomplishing each of the goals, in a unique way. By not mounting the buckles (209) to the socket, and instead allowing them to float along the straps, provides a simplified method of assembling the containment straps, and minimizes wear and tear points. In this embodiment, only the ends of the containment strap (601) themselves may be adhered or connected to the other structural fabric sections of the socket, and in a preferred embodiment, may be attached through very simple and modular velcro-compatible hook or look being sewn to the ends of the webbing, which may be adhered to other velcro-compatible elements of the socket assembly, or to themselves.

An anterior containment strap (208), once assembled onto the socket may create a general loop shape, which may have a size such that the loop only encompasses generally the front half of the BK limb. The adhered ends of the anterior containment strap may be adhered to the anterior panel (202). The anterior side of the anterior containment strap may wrap around the anterior portion of the exoskeleton structure (602), which may be adhered to it, to prevent the straps migration. By doing this, when the posterior containment straps are pulled tight, thereby pulling on the anterior containment straps via the loops, the section of the anterior containment strap that is over the outside of the exoskeleton holds a spatial positioning of that strap assembly, and allows the inner section of the anterior containment strap loop to be pulled backwards toward the posterior aspect of the socket. By doing this, the anterior side of the BK limb (604), by virtue of the anterior panel assembly and anterior containment straps are de-weighted from the anterior end of the exoskeleton (602), which allows the BK limb to rest into a conforming fabric structure (202+), without any rigid structure to contact. The same concept can be accomplished in an above knee socket using its complementary components.

Two posterior containment straps (207), once assembled onto the socket may create each side of a posterior general loop shape. Each posterior containment strap may be adhered or connected with the anterior containment strap loop via a buckle (209), such that as the posterior containment strap is tightened, it may pull on the anterior containment strap via the buckle. The two posterior containment straps may together be joined through another buckle (603), such that they can be pulled again back toward the front of the socket, and connected through a variety of ways, including the possibility of using velcro-compatible hook and loop back to the anterior and/or posterior containment strap's outer surfaces. Alternatively, both posterior containment straps sections may be connected through a tightening mechanism (604) to pull them tighter together. Through either such a mechanism (604), or using the straps themselves (603), a user can self-adjust their prosthetic fit in real-time. As they do, the anterior panel may be generally pulled posteriorly, allowing the limb to be pulled toward the posterior anchors, providing biomechanical connectivity, and increased proprioception. It is understood that the posterior containment straps may also be made of a single strap, which does not anchor to posterior panel(s) 205, but rather floats over the top of them. In such an example, as illustrated in FIG. 6C, the single strap may not require any particular anchor method, except for connecting through anchor mechanism 604, which may allow for a very simple, modular, and adjustable containment means. Further, the routing of such a posterior containment strap may inherently hide the adjustor mechanism 604, as the webbing strap may be positioned over the top of it, thereby creating a more streamlined and cosmetic appearance.

Similar containment strap routing means may be used for above knee sockets as well, whereas the anchored part of the exoskeleton 602 may generally be positioned on the lateral aspect of the limb (versus anterior on the below knee socket fittings), and the posterior panel(s) 206 in the below knee fitting would instead reside on the medial aspect of the limb 205 for an above knee socket fitting.

The structural fabrics may be used to support the limb in certain areas to prevent elongation, to prevent stretch of socket areas, to prevent bulging of the limb in certain areas, and to prevent movement of the residual limb in certain direction and certain amounts. The containment straps provide an adjustable means of allowing the other conforming structural fabric sub-components to form around the limb, regardless of its shape or size, and then provide the appropriate level of containment and support of the limb, thereby further maximizing the connectivity between the limb and the socket. Exoskeleton structure may also be placed around the outside (posterior) side of the posterior panel, and which the posterior containment straps may wrap around.

Instead of forming plastics and laminated carbon fiber and the like, or 3D printed plastics shaped to a static limb shape, this disclosure enables conforming structural fabrics to be tightened around a limb shape, and the structural fabrics conform to such limb shape in real time, causing the structural fabrics to take the shape of the limb shape, versus the limb having to take the shape of the socket shape as in conventional sockets.

Having one anchored exoskeleton support on the anterior side of the BK limb, or lateral side of the AK limb, and having two anchored exoskeleton supports on the posterior side of the BK limb, or medial side of the AK limb, allows the containment straps to generally form a triangular shape from the single support to the dual support structures on the opposing side of the limb, as viewed from a cross section of the socket. The limb is generally pulled toward the dual supports, and the triangle shape that is formed, with the apex of the triangle at the single support. As forces may cause the limb to press into the containment straps between the single support and either of the dual supports, it creates a resistive barrier, preventing limb movement in that direction, as the containment straps are pulled snug around the limb. This provides enhanced biomechanical stability within the socket, yet provides full conformity to the limb shape. With the containment straps tight, the limb is prevented from physically moving past the position of the containment straps, thereby locking the limb in position within the socket. This increases proprioception for the user of the prosthetic position, and increases stability, while not only conforming to the limb, but also preventing it from contacting any rigid structure.

In a similar way, proximal brim-level containment straps may be used to contain and define the brim shape, size, and contouring. While their assembly method and configuration may be different from the lower containment straps, they may serve a similar function, in that they may be tightened by the user to their preferred length for maximizing comfort and limb/ prosthesis control. Their assembly and production patterns may utilize similar look and function as the lower containment straps.

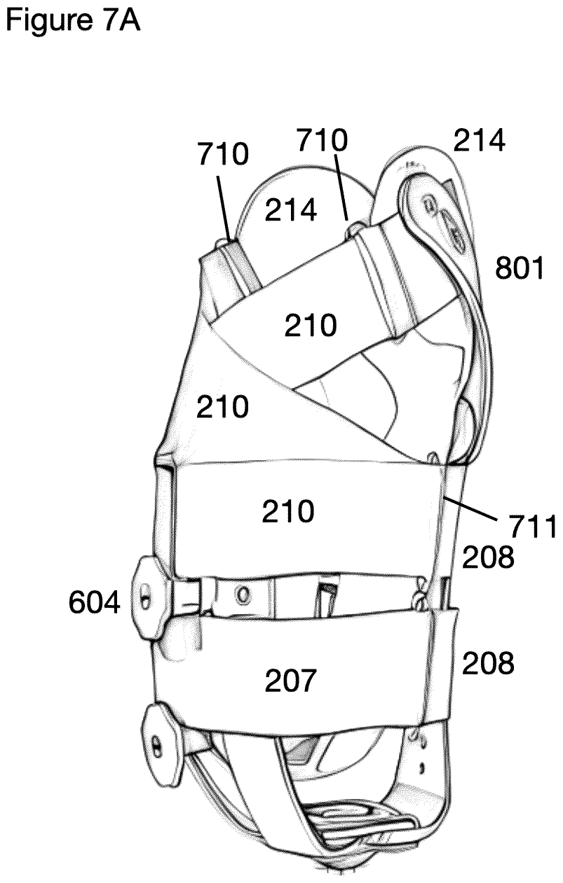

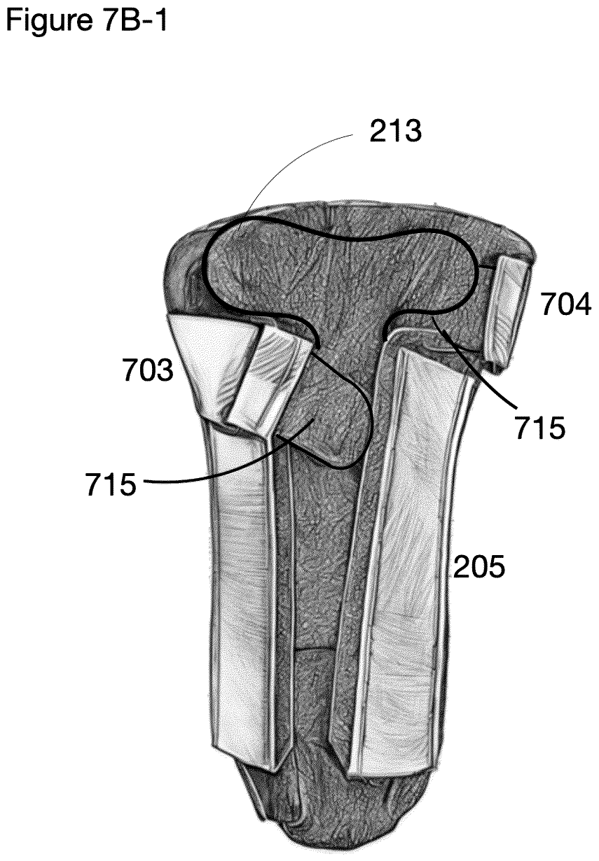

As illustrated in FIG. 7A, in the BK socket configuration for example, the posterior aspect of the brim may utilize a general V-shaped strap assembly using straps 210, which may be able to be tightened by a user, for a customized fit. It's position may be anchored to the posterior panel, and anchored to the sides of the knee areas through buckles, which may be attached to 214. Alternatively, and in a preferred embodiment, the brim straps 210 may be connected to the knee stabilizer panels 214 at their buckle 710, may each wrap around the opposing side of the limb, through an anterior containment straps buckle 208, at their buckles 711, then may loop back toward each other to an adjustor mechanism 604.

In such an example, the brim straps may not directly connect to the posterior panel(s) at all, and allow the height of the brim straps to be higher than the brim of a conventional socket. This may allow the brim to provide increased stance phase stability and biomechanical support of the limb during stance, but since the brim is entirely soft, conforming fabric, it may easily fold into the bend of the knee during flexion or sitting without impingement or restriction.

This may put them in a position to slightly overlap the posterior brim panel(s). The posterior brim panel may be made of conforming materials, which may include foams, leather, structural fabrics, and/or the like. The posterior brim's purpose is to provide biomechanical stability of the limb within the socket, while allowing conformity during knee range of motion, so that it folds with the fold of the knee, to increase comfort during knee range of motion. To assist with this, the V-shaped strap assembly 210 may slightly overlap the brim panel, so that during the stance phase of gait, when the limb is relatively straight, the V-shaped strap assembly may support the brim pad and provides increased anterior-posterior limb support by maintaining the height of the posterior wall of the socket, to be at least nearly equal to the height of the anterior brim. However, during sitting for example, when the knee if flexed, the relative position of the V-shaped strap over the brim pad may slightly shift, allowing for the brim pad to fold to the knee flexion angle with minimal resistance, effectively lowering the posterior brim, and allowing the posterior brim's shape to conform to the limb shape, thereby increasing comfort for the wearer. The flexion point may be along the proximal area of the posterior panel, whose contouring and the conformity allows for a living-hinge in that area, through the structural fabrics connection area.

As illustrated in FIG. 7B-1, in a similar way the AK brim shape and contouring may be controlled by overlap of the AK brim straps 703 and 704 and how they overlap the AK brim pad 705 in the areas of 703 (anterior medial) and 704 (posterior medial). These may be positioned to allow the AK brim pad to flex during range of motion, and during weight bearing, as only a portion of the AK brim pad may be supported by the brim straps during weight bearing, to allow for increased comfort in areas that can benefit from increased conformity. The amount of tensile support of the brim pad, via the AK brim straps, provides the realized and/or perceived amount of rigidity versus flexibility in such areas. Therefore, brim areas that can benefit from increased rigidity or tensile support may be more directly connected to the brim straps to provide increased support (such as the ischial tuberosity area, in area 704), whereas areas that could benefit from increased flexibility may not be directly connected to the brim straps (such as the medial and anterior end of the medial brim, in area 703), thereby allowing them to have more flexibility as the brim pad 213 may slide past the brim strap, which is connected to its anchor point 715, though may be supported during certain range of motion movements. The anchor points (715) of the two brim straps may be modularly customized or positioned such that the anterior and posterior brim straps provide the correct amount of brim support, support direction, and brim dynamics as desired by the user for optimal comfort. For example, they may be placed or mounted more proximally or more distally with respect to the brim of the socket, thereby providing less or more actual brim support along the medial brim, depending on their position and resultant placement of the tensile support. The other ends of such brim straps may be connected to the lateral assembly and exoskeleton support, 212, thereby creating the effect of a circumferential loop around the limb, with at least one adjustable means, but preferably two. It should be noted that depending on the positioning of the anchor points 715, the brim straps may not actually create a full circumferential loop, and in a preferred embodiment, won't. The area in between, which may generally be considered the medial brim of the socket, may not include a brim strap material at all, and that area may only be supported by other structural fabric panels, which may be connected to the mounting 715 of the brim straps. This is an important distinction of this disclosure, as the medial AK socket brim is now able to be modularly managed in its comfort by way of the modularity of the brim strap connection points and resultant brim support, brim forces, and brim comfort.

As illustrated in FIG. 7B-2, the mounting points of the brim straps 211 may be connected to brim panels 205 at or near the brim level. Such brim panel 205 may have a buckle in which a brim strap 211 may route through, thereby creating a modular and adjustable mounting point. Each brim panel may utilize conforming material, and may use a stiffening material 712 attached thereto to prevent collapsing of the brim panel. Such stiffening material, 712, may generally be flexible in twist/torsion, bendable in at least forward and backward, but not collapsable in the length direction. Since the containment straps, and brim straps, prevent the brim panel from ballooning outward under load, and the limb prevents inward movement by pushing outward from within, the stiffening agent prevents buckling, and therefore the medial aspect of the socket can be compliant, conforming, accommodating for limb shape and size, and yet still provide full limb support through using soft compliant materials in combination with each other.

Through the above description, it should be obvious to one familiar with fitting prosthetics that connecting the various structural fabric sub-components together using methods such as velcro-compatible materials, allows for an expedited fitting process. In practice, using these methods and sub-components, an amputee should be able to be fit and walking on a prosthesis within 30 minutes to an hour from start to ambulation. While there may be further adjustments, and general socket alignment to address thereafter, being able to go from start to walking on a prosthesis within such a short period of time is unheard of in the field of prosthetics. This speed has been demonstrated in practice, resulting in an ultra-comfortable prosthetic socket. More importantly than the speed, this method of fitting is uniquely simple enough for a layman to accomplish along with simple instructions. Using structural fabrics, and more specifically using velcro-compatible structural fabrics, as a means of connectivity assists in the expedited nature of this type of prosthetic fittings, as the various structural fabric sections can literally be placed on top of the others in the correct positions, thereby connecting them together in a near-permanent manner in seconds, without the need of any tools or traditional fabrication methods.

Only a simple set of tools would be used in this described fitting method. Scissors would be used to cut various structural fabric sub-components to length such as the sleeves or panels. Allen wrenches would be used to modify the lengths, angles, and positions of the various exoskeleton components, and for conventional socket alignment principles. It is possible that no other tools would be required to fit or fabricate this type of socket to an amputee.

Because this socket is made of structural fabrics and is modular in nature, if any sub-components were to wear out or break they could be easily replaced by a layman. The conforming nature of the structural fabrics also makes them inherently more durable than conventional rigid materials, because under high load conditions, the socket materials are able to flex and give, dispersing the forces. This also greatly improves comfort and protection of the sensitive residual limb skin. In conventional sockets, the limb is pressed against a rigid socket structure, which forces high loads in concentrated areas within the socket.

Unlike conventional sockets which can only be wiped out, the structural fabric flexible inner socket can be removed from the exoskeleton structure and put in a washing machine, providing a more thorough washing and hygiene.

### Exoskeleton Structure

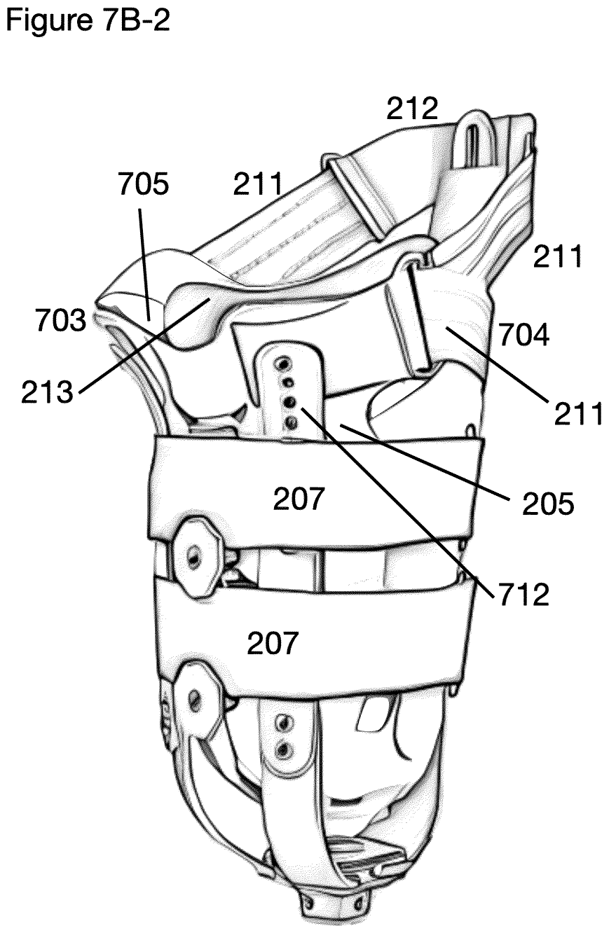

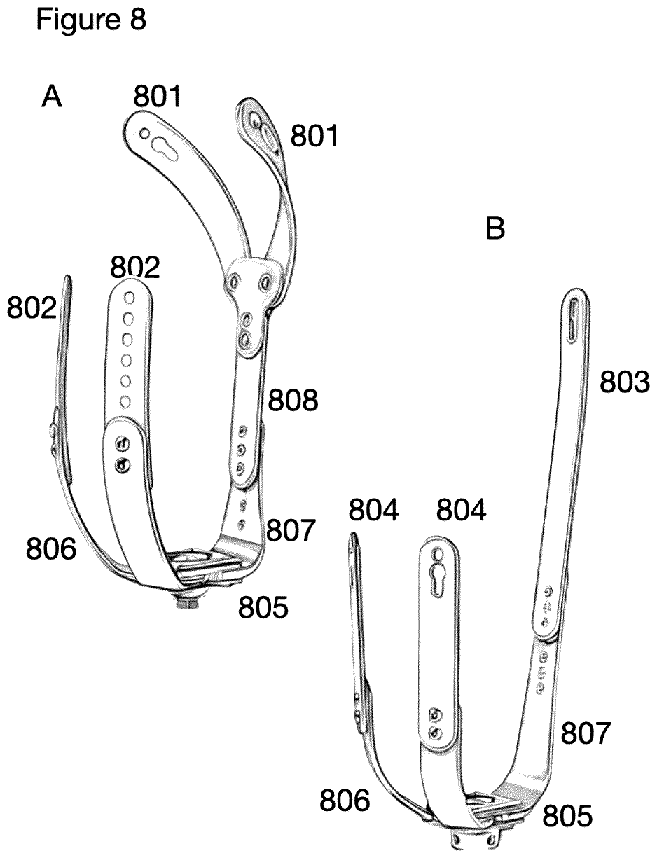

As is illustrated in FIG. 8, the exoskeleton structure may include a common structural base area (805), structural extensions from the base unit such as 806 and 802 for the posterior side of the BK limb, and 806 and 804 and 802 for the medial side of the AK limb, a long structural extension spine 803 for the lateral side of the AK limb, and knee stabilizer structural extensions 801 for the proximal medial and proximal lateral side (via the anterior 808) of the BK limb. Each of these structural extensions (802, 801, 802, 804 and 803) may be used to connect to the structural fabric sections to support the flexible inner socket, and hence the limb. Likewise, other means of connectivity may be used, such as but not limited to anchoring with screws, snaps, buttons, or any number of other common methods used to connect fabric to structure.

In a preferred embodiment such structural components may generally be rigid to best support the limb, though component 802 may be flexible, though preferably non-compressible, to better match the limb's contouring.

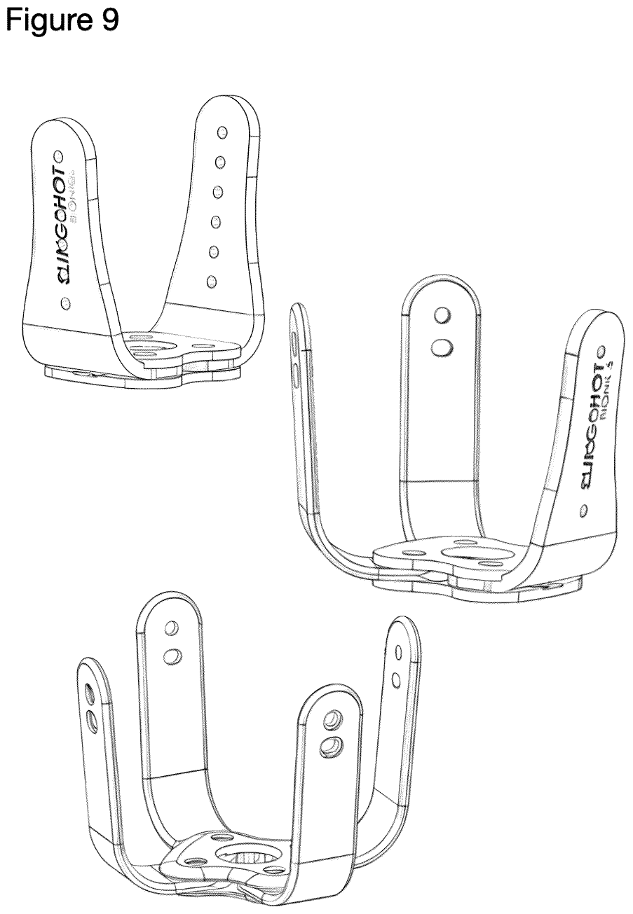

The size and positions of the various exoskeleton structure sub-components may be modularly adjusted. For example, the base area may allow for various numbers, sizes, or angles of base extensions (806) to extend out from it, such as two, three, four, or other (as illustrated in FIG. 9) and each may have a different amount of curvature, as illustrated in FIG. 10, and hence cumulatively may provide a different amount of space in between for the limb to be contained. FIG. 10 illustrates an overlap showing perspectives of various sizes, bend angles, and resultant space for the limb, of various extension configurations that may be mounted to a base. Each base extension may have an adjustable length extension (802, 804) extending from it, which may be adjusted in length or connection to provide the correct span from the socket's medial brim level to the base (805) for AK, and from the socket's posterior brim level to the base (805) for BK. Sub-components 802 and 804 may have numerous holes to allow for modular length connectivity to 806.

In a similar way, the other side of the exoskeleton structure may as well offer length adjustment, such as sub-component 807/803 connection for AK, and 807/808 connection for BK. Since this side of the exoskeleton resides on the opposing side of the limb as the medial brim (AK) and posterior brim (BK), the length adjustment of 807/803 and 807/808 provides adjustability of the anterior brim height (BK) and lateral brim height (AK). In a preferred embodiment the lateral length adjustor of the AK socket, 803, may include a curved proximal end, or may offer a modularly attachment component which may attach to it, which may have a curvature, to follow the shape of the limb.

It is understood that the span or length of the knee stabilizers 801 may be adjustable, or may be swappable for longer or shorter versions. However, it has been found in practice that one or two sizes of knee stabilizers 801 will fit the vast majority of limb sizes, as the angle between the knee stabilizers may be adjustable, and hence allowing the proximal end of the knee stabilizers to be positioned closer together or further apart. The knee stabilizers may be formed with a preset amount of twist and bend, to generally contour around the knee shape, though that one common bend and twist shape may accommodate for the vast majority of limb shapes, thereby making it modularly acceptable for most users. The proximal end of the knee stabilizers may be positioned generally near the center of the knee joint, and the distal end of the knee stabilizers, depending on their respective angle, will fall higher or lower along the length of the limb depending on their angle, and along the front of the limb. The knee stabilizers may have a predetermined amount of both curvature and twist, allowing their shape to transition from originating from the front side of the limb, to terminating on the lateral and medial sides of the knee. As such, the length of 808 may be adjusted, or its connection point on 807 may be adjusted to create the overall length between the attachment end of 801 to the base 805. This allows the proximal ends of the knee stabilizers to be comfortably positioned against the sides of the anatomical knee, to provide increase stability during ambulation, and the proximal to distal socket length can be separately adjusted via 808 and 807.

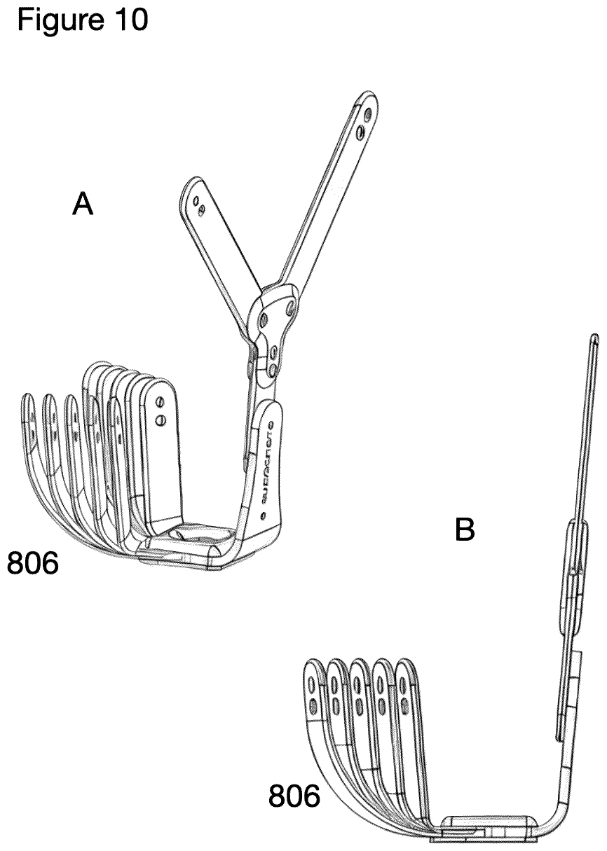

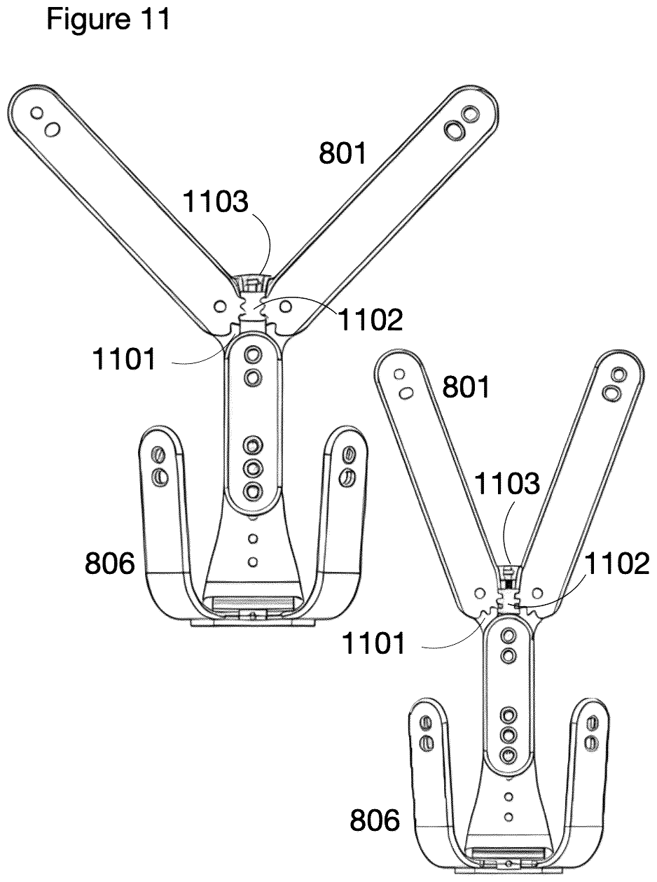

The angle adjustment between the knee stabilizers can be accomplished through various active or passive means. In a preferred embodiment, at least one end of the knee stabilizers may have gear teeth, or equivalent (1101), to cause the two to move in coordination with one another, as illustrated in FIG. 11. The distal end of both knee stabilizes may directly engage with each other, or may engage with each other indirectly, as by example with another gear in between (1102), as illustrated in FIG. 11. In a preferred embodiment, a squiggle gear 1102 may be placed in between the two knee stabilizers, and which engages with the two knee stabilizers. Such squiggle gear may have a threaded screw engaging within it, and the end of the threaded screw may be captured in position within a housing area 1103, to prevent the head of the screw from moving proximal or distal. Since the screw's position may be locked relative to the position of the knee stabilizers, as the screw is turned, the threads thread into or out of the squiggle gear, forcing the squiggle gear to move proximal or distal. As the squiggle gear may move proximal or distal, with respect to the screw head, it may cause the knee stabilizers to rotate about their connected axis, thereby changing the relative angle between the knee stabilizers. This method effectively creates a non-back-drivable gear system. This allows for a simple and ultra-low profile user-initiated adjustment of the knee stabilizers angles by the simple turn of an Allen wrench, for instance, by turning the screw in or out. Regardless of the knee stabilizers being directly or indirectly connected together, so that they move in coordination with each other, provides increased structural stability of the knee stabilizer pair, and provides increased biomechanical stability of the user during ambulation. In a preferred embodiment the knee stabilizers respective angles may be positionally lockable, to prevent their movement during ambulation.

This locking may also be accomplished through the structural fabric dynamics, which is connected to the knee stabilizers, or may be accomplished by mechanical means, for example. A structural fabric's locking means may be accomplished through the knee stabilizers being connected or affixed to the knee stabilizers panels, which may ultimately adhere to the anterior panel 202, which may create a connected and defined span length in between the ends of the knee stabilizers. Therefore, the distance between the ends of the knee stabilizers may be prevented from moving further apart during ambulation because the connected structural fabrics may span between them in a way in which the structural fabrics can not move further apart, especially when the human limb is pressed against the structural fabric assembly.

Alternatively, other mechanical lock methods may be accomplished through any number of locking means, however, in a preferred embodiment, this lock should allow for an infinite number of lockable positions, be minimal in size and weight, and be durable.

Alternatively, instead of the screw threading into the squiggle gear and directly driving the squiggle's position, the screw may instead be threaded into a base, at or near 1103, allowing the screw to move back and forth with respect to the base as it threads in and out, and based on the distance that the screw is threaded into the base, and extends through the other side of the base determines how much space is left for the squiggle gear to move within a designated track or area. This method may allow the knee stabilizers to be able to lock from moving outward, or away from each other, but not inhibit them from moving toward each other in angle. This provides dynamic motion in one direction but lockable in the other. In the use-case of a BK prosthetic socket for example, preventing motion in the outward direction is important for the user's biomechanical stability, while the anatomical knee by itself prevents the motion inward.

However, when the device is not being worn, it may be simpler to slide the knee stabilizers into their respective sleeves or mounting areas if they are allowed to move inward toward each other. The knee stabilizers, through such a system, may provide a uniquely customizable solution for contouring the proximal ‘ears’ of a BK prosthetic socket to the anatomical knee shape. The knee stabilizers' twist and bend amount may be suitable for the anatomical knee shape, regardless of patient size, going from the anterior area of the BK limb near the tibial tubercle to the center axis of the knee along the medial and lateral sides of the knee, to the posterior aspect of the knee, where the knee stabilizer panels terminate on the posterior side. The knee stabilizers by themselves may provide a relatively small surface area of contact with the sides of the knee, which may provide adequate structural support to the sides of the knee. However, in order to disperse the forces for added comfort, the knee stabilizer panels or connection areas 214 may utilize a broad section of connected structural fabric which may contour to the broader knee shape, thereby dispersing the forces from the knee stabilizers' support through a broad area of the knee. Such structural fabrics may be self-contouring to the knee shape, as the structural fabric assembly effectively hangs from the knee stabilizers, via their connectors. As such, the brim strap assembly provides contoured support from the knee stabilizers, around the posterior aspect of the limb. The tightness of the brim strap literally presses inward against the knee stabilizer panels, forcing them to take the shape of the limb shape, and therefore the socket's shape mimics and contours to the limb shape in real-time, thanks to the conforming and adjustable materials that make up the socket. Likewise, the structural fabric connected to the knee stabilizer panels may extend around the front of the limb, as they may connect to the anterior panel, which then may create a bridge between the ends of the knee stabilizer panels to support the anterior aspect of the brim. In effect, the entire brim, anterior and posterior, may be supported by the knee stabilizer assembly, which self contours to the limbs shape, increasing comfort.

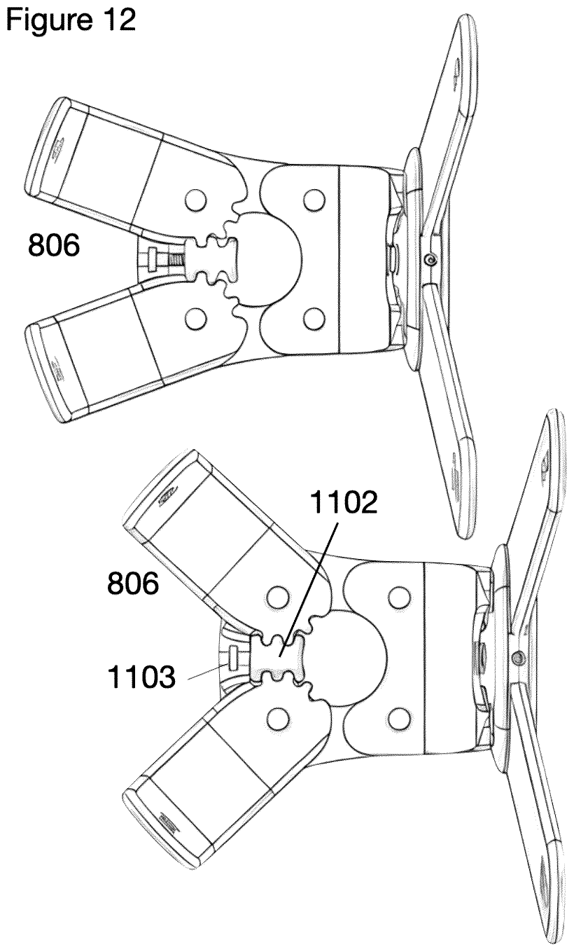

Similar means as described in FIG. 11 may be used at the base to adjust the angle between various base extenders 806 as illustrated in FIG. 12. This may assist in locking the angular position of the base extenders, which may be helpful for contouring to the limb shape, which may accommodate for the limb's circumference to fit within the exoskeleton.

Still further, similar means as described in FIG. 11 may be used to generally contour to and lock position of structural components around the upper extremity limb, and which may contour around the below, instead of the knee, or around the shoulder, instead of the knee. Such structure may also be used to capture contouring proximal to the elbow condyles, to provide suspension, with attached compliant straps and/or structural fabrics, not too dissimilar to how it is described in the BK or AK scenarios.

### Exoskeleton Orthotics

Still further, similar means as described in FIG. 11 may be used to generally contour to and lock position of structural components around various aspects of the body, including as a general connection of components around the hips, as they may connect with a spine segment of an exoskeleton. In such an example, the spine segment may be connected to the knee-stabilizer-equivalent parts, but for contouring around the hip joints. Their joint may utilize the adjustable mechanism described for use with the knee stabilizers, or may utilize other attachment means, such as positional anchor points. It may then connect to hip joints, which may connect to other sub-components such as a joint, as an extension to other sub-components connectivity.

Since there is a vast number of the global population with ambulatory issues, including amputation but also other non-amputation related ambulatory issues, using the sub-components in these descriptions for other use-cases besides a prosthetic socket allows for a simple set of sub-components to be applied to benefit various forms of ambulatory issues. All use-cases share the need to contain limb tissue, control limb tissue, and/or establish biomechanics control of the limbs within or with a device or apparatus. Therefore, similar means may also be used to contour around and lock position around other limb segments, including for exoskeletons, orthotics, braces, and the like.

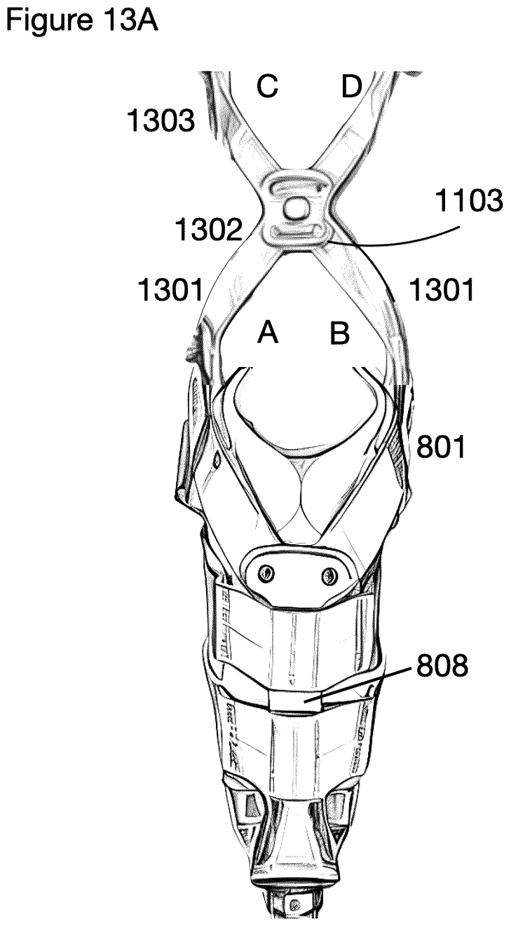

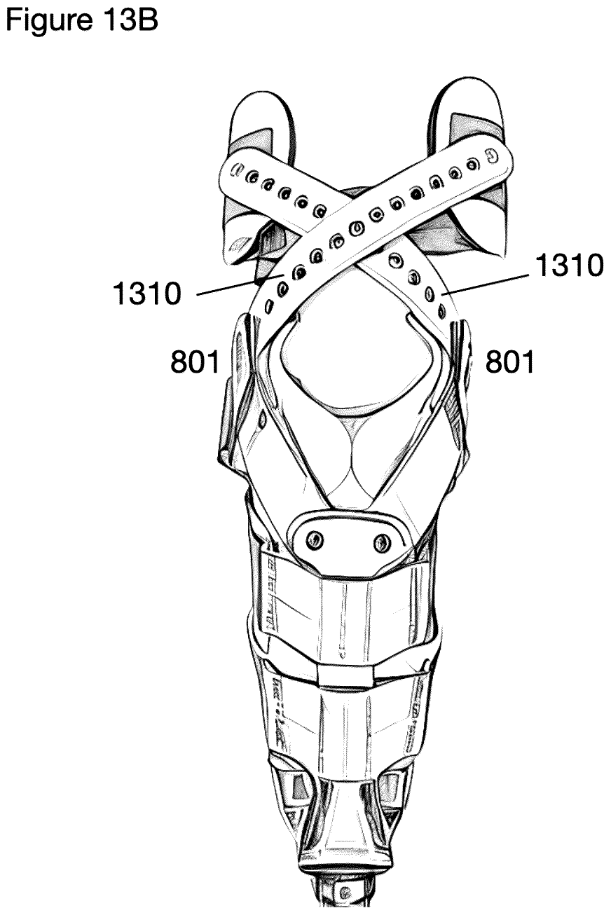

FIG. 13A illustrates an embodiment of a knee brace which may be used in coordination with a BK prosthetic socket for suspension aid for example. In such a use-case, the knee brace may connect with the knee stabilizers proximal end, 801. Another set of knee-stabilizer-like curved and twisted components may be use, 1301, which may generally attach at or near the proximal end of the knee stabilizers, effectively creating a joint at the proximal end of the knee stabilizers. Such joint may use gears, similar to those found on the distal end of the knee stabilizers, to allow them to move in coordination with each other. The proximal end of 1301 may also have such a gear system, similar to 1103, as has been described for the locking of the knee stabilizers, thereby allowing for their respective angle to be modularly adjusted. Sub-component 1302 may function similar to area 1102 and 1103, allowing the angles of 1301 to be dialed into a desired angle. Sub-component 1302 may offer a dual sided connection, allowing yet another set of similar knee-stabilizers-like components, 1303, to extend again from it, generally forming an X shape. This X-shape may be generally placed over the anterior side of a thigh, and may offer an adjustable strap system attached therefore and positioned across its posterior side, which may allow the system to be tightened around the limb shape. Such a strap system may generally connect to the various 1301 and 1303 components to connect together, and may generally be in a similar X-shape along the posterior aspect of the limb, in a preferred embodiment. This X-shape advantageously may not put pressure near the fold of the knee and therefore not prevent range of motion of the knee. In such a system, a knee brace may be created which is modularly adjustable in width toward the top separate from the width toward the bottom, to match to the desired thigh dimensions.

And further, the shape and positioning of such a strap system may provide contoured contact pressure proximal to the knee condyles, thereby providing a means of supra-condylar suspension for a BK amputee. Such straps may be adjustable, conforming, and supportive. As is illustrated in FIG. 13A, in a preferred embodiment, such a strap may generally be mounted near point A on sub-component 1301, and extend around the back of the leg, to near position D. A similar strap may extend from B to C. The positioning of anchor points at A and B may allow for the strap to generally pass just proximal to the femoral condyles before wrapping around the backside of the limb. An additional structural fabric pad may be placed on such strap, which may have a relatively stiffer characteristic, allowing for it to press into the body's soft tissue above the condyle, thereby providing a more secure lock over the condyle. If the pressure or compression or tightness proximal to the knee condyles provides a smaller size capture than the condyles size, and is therefore not able to move distally past the knee condyles, the knee brace structure may prevent the BK socket from moving distally, and therefore provide suspension to hold the prosthetic socket in place.

Unlike conventional supracondyler socket suspension systems which are static and rigid, the conformity and self-adjustability that this design offers is advantageous to the quality of life of its users. Likewise, conventional BK corset style suspension methods can be difficult to fabricate and they trap heat. While it is common for below knee prosthetics to be fit with gel liners with pin systems, vacuum or suction suspension in the developed world, the majority of the global amputee population, and especially those in developing nations do not have access to a gel or silicon liner, as they are largely inaccessible. As such, there are not other existing comfortable and secure methods of suspending such a prosthesis, and this disclosure provides a technical and comfortable solution.

FIG. 13B illustrates another embodiment for supracondular suspension, but using more compliant materials versus rigid structure. In such an example, non-compressible, yet bendable straps 1310 may be used to connect to 801, which may then connect to structural fabric panels, which may incorporate an adjustable strap system. The structural fabric panels may compress into the soft tissue above the knee condyles once the straps are tightened. In such a system, it may prevent distal migration of the socket, and hence suspension.

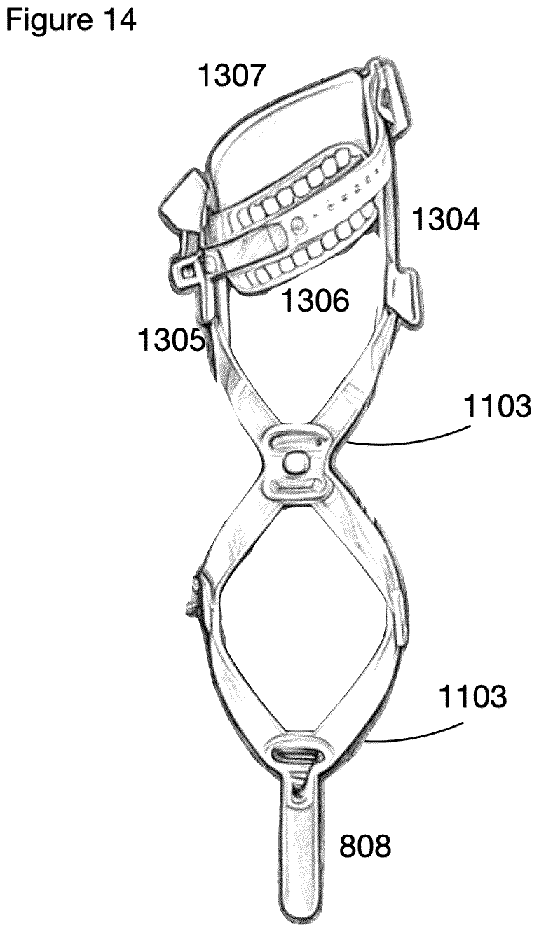

FIG. 14 illustrates a similar embodiment as FIG. 13, except that it may add a proximal brim, proximal to sub-components 1303. Such a brim may be mounted to 1303, or may be mounted to extensions (1304, 1305) that extend from 1303, thereby providing a simpler method of adjustability. Regardless of the height adjustability mechanism, the proximal brim may generally utilize an anterior 1306 and posterior 1307 aspect. Such a brim may be adjustable in length, and may provide conforming support of the body in the groin area, while suspension of such forces from 1306 and 1307. The brim components 1303 may include various tightening mechanisms, containment straps to form the posterior and medial brim areas, and the like, and may use any combination of structural fabrics to form the general brim area, similar to the prior descriptions. Through this integration, a similar set of sub-components as has been used to construct the AK and/or BK sockets, can have alternative use-case to extend the physical man/ machine interface beyond the prosthetic socket, and distribute forces about other limb segments or use-cases. This may be used for example for a BK amputee who desires to de-weight their residual limb due to sensitivity, or who would prefer added knee stability. By doing this, the weight of the user may be bore at least partially through the brim, at the thigh level, de-weighting other parts of the limb. This may be especially helpful for an amputee, for example, who has limb sensitivity and can not bear weight in their residual limb. The limb may still reside within the socket apparatus, but such apparatus may more generally be used for spacial positioning of the limb, versus weight bearing support.

Still further, such a configuration, as illustrated in FIG. 14 may connect in an alternative method to 808 as a use-case as a prosthetic socket support structure, and instead may extend past 808 to the ground without a socket for an amputee. Such exoskeleton structure may be used as an orthotic device, foregoing at the least the distal umbrella structure from FIG. 5, thereby allowing the intact limb to extend through the interface to the ground. This may or may not include all or some of the other structural fabric sub-components described herein, but any of which may be used for that particular use-case to capture and control the limb segment though an orthotic or limb-exoskeleton use-case.

In such a case, it may be used as an exoskeleton orthotic, for an amputee or non-amputee. Section 808 may generally extend around the lower limb segment, and may utilize an interface similar to shin-guard-like assembly, not shown in the illustration, which may help connect the general area 808 to the lower limb for positional control. Likewise, it may use the described anterior panel just the same. Such a configuration may provide the ability to bypass weight bearing of the below knee portion of an intact limb, or bypass weight bearing of the knee area of an intact limb, for use-cases of a person with an ankle or knee injury. The knee joint area (801 to 1301 connection) may offer a lockable means, or may be allowed to move in coordination with the intended knee movement. Regardless, such a system may be applicable to those who otherwise may rely on crutches or other similar assistive devices, but may provide a hands-free alternative.

It is understood that similar structural fabric methods as described herein may be used to provide contouring, security, control, fit, or other such attributes of the limb to the exoskeleton structure. In a similar means as described in a prosthetic use-case, various structural fabrics may be utilized around the limb to capture, control, and/or contain the limb tissue, which may benefit the biomechanics of ambulation.

### Alternative Suspension Methods

Every prosthetic requires a method of holding the prosthetic onto the limb. In conventional sockets, one of the most common and secure methods to hold the limb into the socket is through using a gel liner on the limb, and the end of the liner may use a pin lock or lanyard which engage into a lock mechanism on the prosthetic. The issue with this is that the majority of the global amputee population does not have access to a gel liner, and so this disclosure benefits from the ability to not have to use a gel liner. Pin systems and lanyards often have challenges with pistoning and rotation issues in conventional rigid sockets. However through using conforming materials, a gel liner with a pin or lanyard system works very effectively because the conforming materials contour so effectively to the limb, thereby preventing pistoning or rotational issues.

Conventional pin locks can also be heavy and bulky. Additionally, lining up the liner's pin with a conventional lock mechanism mounted on the rigid socket can be challenging. In a conforming socket that challenge may be compounded, as the limb's position may not be in alignment with the center of a structural socket, and hence may not line up with the center of the pin lock mechanism, which may be centered in the structural center of the socket. Therefore, an alternative suspension method is needed. Rather than lining a pin up with a hard-mounted lock, in a preferred embodiment, the lock mechanism may line up with the position of the limb/liner/ pin.

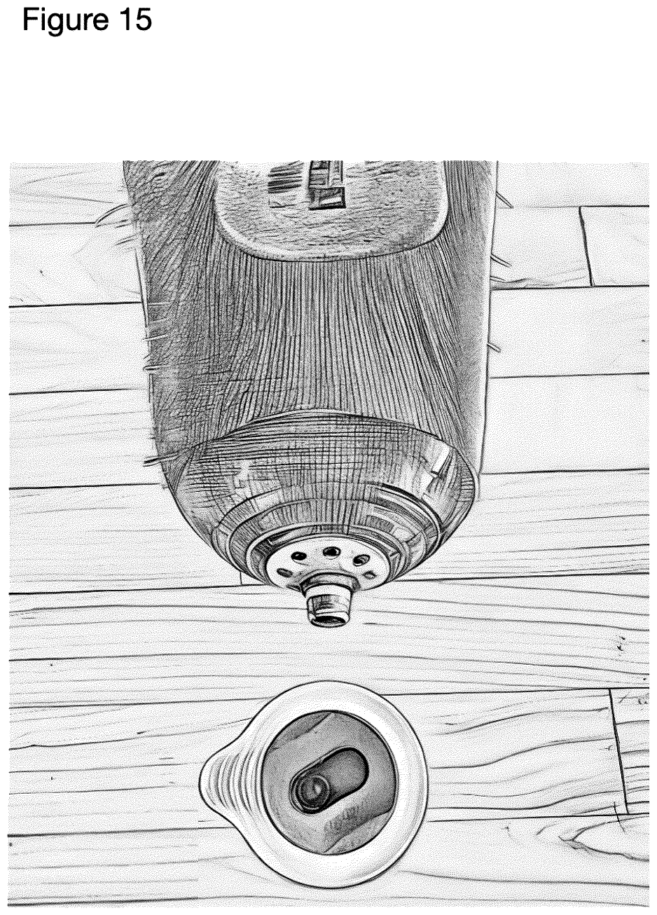

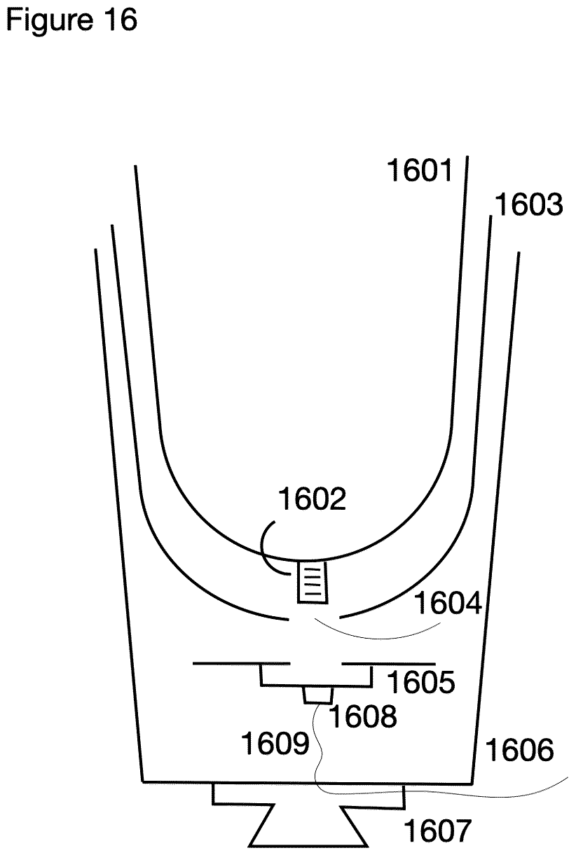

As illustrated in FIG. 15, as the liner (1601) is donned on the limb, the limb and liner are then donned into the open, conforming, and loosened socket (1603), the pin (1602), for example, may easily protrude though an opening (1604) in the distal end of the socket. A lock mechanism (1605) may be clicked onto the pin system (1602), though the lock mechanism may not otherwise be rigidly connected to the socket. If the hole that the pin pushed though (1604) is smaller than the lock (1605), the lock will not be able to go back through the hole, and hence as the limb tries to pull upward, the lock mechanism may hit against the bottom of the socket and result in suspension or security to prevent the limb from extending out of the socket. In such an example, the socket (1603), in this case a structural fabric flexible inner socket, may be mounted to an external frame or exoskeleton structure (1606), which may be connected to mounting hardware (1607) for attachment of the prosthetic foot or knee for example. Therefore, there may be space between the end of the socket (1603) and the end of the frame (1606), thereby allowing space for the lock (1605) to reside within and have space for a user to reach in and lock and unlock such unit from the socket.

It should be noted that this is distinctly different from a conventional socket where the lock is integrated within, and there is no space and no access point for positioning a lock in between an inner socket and a frame in conventional fitting methods. It is understood that with unique fabrication methods, a space could be built into a conventional rigid socket, with a large enough opening, whereas a conventional inner socket (typically plastic) could be positioned to secure a lock distal to the inner socket but above the bottom of the conventional socket frame in a similar manner.

Various embodiments of lock mechanisms may be used. Any such lock mechanism may engage with a standard commonly used pin lock that extends off the liner, or may be a unique pin-like shape or concept. While a standard off-the-shelf lock could be used in this application, a unique lock mechanism is contemplated herein. Off-the-shelf locks are intended to be rigidly mounted to the base of the socket, and the pin is supposed to extend down to meet it. In this disclosure however, the opposite may be true. The off-the-shelf lock may not be mounted within the socket, but instead may float, only connected by the pin itself. As a result the liner and limb may be on one side of the socket (1603), and the lock mechanism with connected pin is on the other side of the socket, though neither may be directly connected to the socket. In this case it may be a structural fabric socket, though a conventional rigid socket may work as well if there is space to allow such a lock to be positioned below the end of the socket and the mounting point of the socket to the components, in the fabrication.

In a preferred embodiment, a common pin lock mechanism, or the like, may be used, as the pin's placement into the lock may be modularly positioned in length, so as the limb pushes deeper into the socket, and extends further through the distal hole, the lock may be pushed upwards more to more fully engage with the pin's length. Alternatively, since the socket's containment and hence tightness is able to open up loosely for donning, there may be a single position, as in length, for the ‘pin-like’ part to engage with the lock mechanism.

In a preferred embodiment, a magnet or magnetically engaging material may be placed on the end of the liner, and either a magnet or magnetically engaging material may be integrated as a functional part of the lock, so that when they come in proximity to each other, the magnetic engagement may draw them together, and a mechanical latch may engage, thereby securing them together.

It is contemplated that an optional lanyard-like system may similarly be integrated as well, which may also use a magnetic latch system to assist in drawing the lanyard to connect with the liner. Such a lanyard may be attached to the magnetic latch (1608) and then a lanyard cord (1609) may extend through a portion of the socket's base to create an anchor point, thereby drawing the limb downward as the lanyard is tightened.

Whether a pin-like system, a lanyard-like system, a magnetic lock, or any combination thereof, allowing the mobile limb, to engage with a mobile lock benefits the user by not having to line up their limb with a rigidly connected lock mechanism. This allows for greater comfort, as the liner and pin will no longer be torquing on the end of the sensitive residual limb as is commonly experienced with conventional hard-mounted pin systems.

FIG. 15 illustrates a top down perspective of an embodiment of such a mobile lock mechanism, using a magnetic connector. In the illustration and gel liner is shown with a magnetic component attached to its distal end, which may be designed to engage with the mobile lock mechanism. Such a lock mechanism, in this embodiment, may therefore be extremely low profile and lightweight.

Prosthetic suspension methods are used to prevent the limb from slipping out of the socket. However, it may also be helpful to prevent movement of the limb within the socket. During ambulation there are various micro-movements that take place in a socket, such as rotation, micro-pistoning, micro-slipping, and the like. Reducing or eliminating such micro-movements can make the prosthetic feel more secure to the user.

Kota (U.S. Pat. No. 8,142,517 B2) described a method of securing a limb within a prosthesis using a uni-directional textile-based material. Using a micro-movement-eliminating modular component to benefit this described socket apparatus would be beneficial. However, it has been found that instead of using a synthetic woven material as Kota has described, that using a more commonly available fur hide material may be more suitable for securing contact with off-the-shelf gel liners. As such, in a preferred embodiment, a fur hide, such as from cow or other animals, may be integrated within a socket, and in a preferred embodiment, may be placed on the inside of the described anterior panel and/or posterior panel, or on the inside of the velcro-compatible sock, or in other areas, to interface with the surface texture of a gel liner, which may be donned within. By doing so, the friction interface reduces micro-movements within the socket, and assist in holding the prosthetic in place.

### Adjustor Mechanism

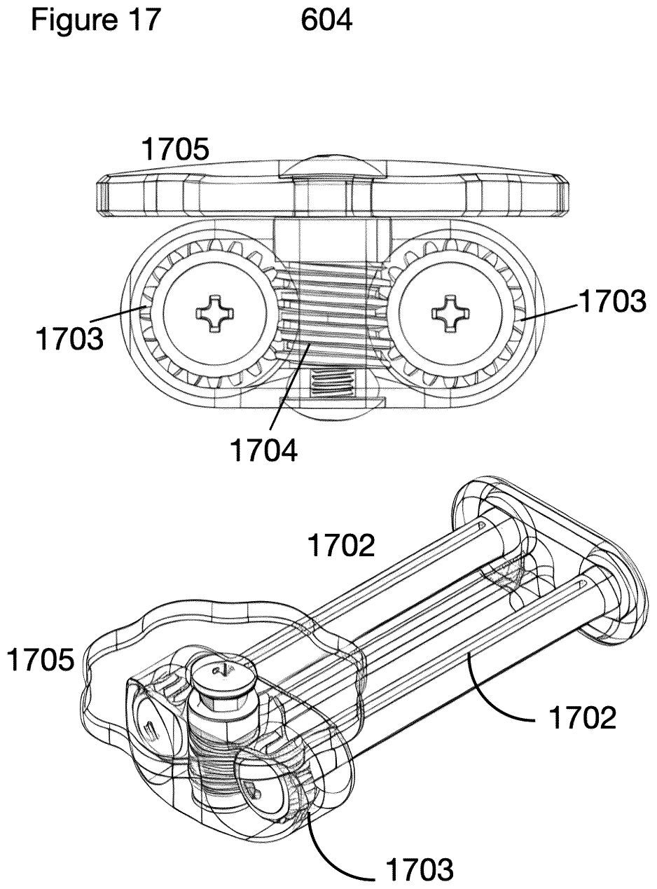

FIG. 17 illustrates an embodiment of an adjustor mechanism 604 which may be used to dial in precision tightness of a structural fabric webbing of such containment straps. In such an example, the structural fabric webbing may be modularly placed in position through slots in shafts 1702. Such shafts may be connected to gears 1703. Such gears may be connected to other gears 1704, which may allow for a more ergonomic manual dial 1705 to be integrated for adjustability. Such as system may be low profile and may offer mechanical advantage for tightening the containment straps. It is understood that other adjustor mechanisms may be used as well to accomplish the same.

In such an example, the webbing may be routed through slots in the shafts, and pulled to a sufficient tightness around a limb. As the dial mechanism is turned by hand, it may turn a series of gears, thereby causing the shafts to turn, and hence roll up the webbing to a desired tighteness. Such gearing system may leverage a mechanical advantage to tighten the webbing, and a lockable mechanism or means to prevent unrolling of the webbing.

Such an adjustor mechanisms does not require mounting, and may generally float and self-align over the socket configuration. This may allow for ease of assembly, modularity, and a lower profile method for improved cosmetic appearance.

### Modularity to Use with Conventional Prosthetic Sockets

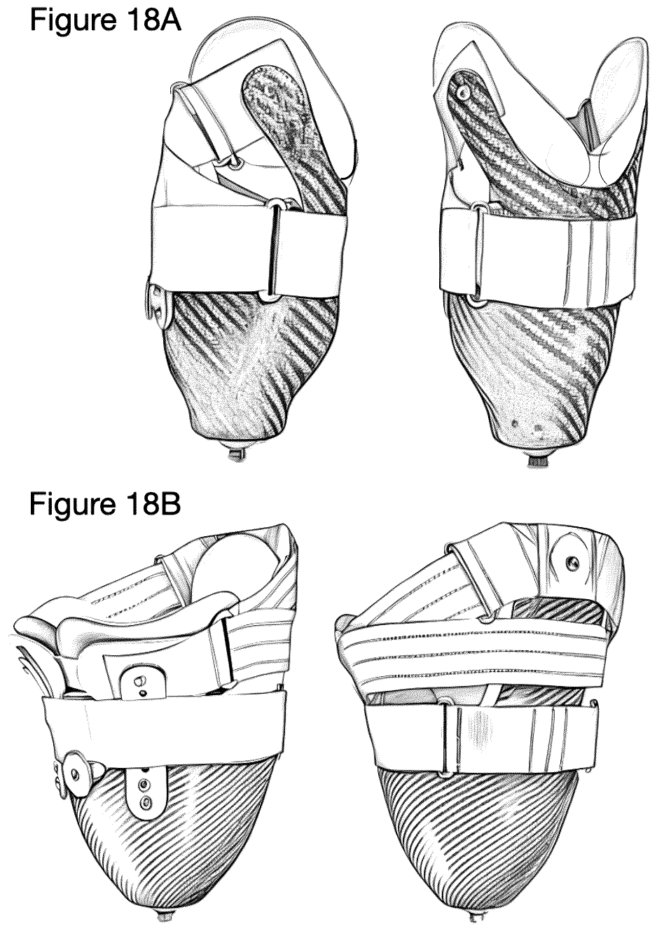

A particular advantage of the described modular prosthetic socket is that the various subcomponents may be assembled in various configurations to accommodate for the wide variety of residual limb needs. As such, while the described configuration allows for a full prosthetic socket to be assembled without the need for any conventional fabrication, it is conceived that a combination of conventional prosthetic socket with the described modular structural fabric socket components may be advantageous for certain amputees. For example, some residual limbs may benefit from the familiar containment of the distal end of a conventional prosthetic socket, while the top half may benefit from the conforming and adjustable fabric-socket elements as described. As is illustrated in FIG. 18A (BK) and 18B (AK), a portion of the top section of a conventional prosthetic socket may be trimmed off, and that section may be replaced with the modular structural fabric components, as described previously. Once assembled, the resultant prosthetic socket may still encapsulate a similar portion of the residual limb as the original rigid socket, but the replaced elements would allow for conformity, adjustability, and hence greater comfort than the original rigid counterpart. The trimming of the conventional prosthetic socket could range from just replacing the brim area, to all the way down to replacing everything except the very distal end of the conventional socket.

Another advantage of doing this is that it could be integrated into an existing conventional socket, and maintain the existing alignment of the original prosthetic.

In the example of FIG. 18A, and by not extending the anterior panels (BK) and medial panels (AK) all the way down toward the bottom of the limb length, the knee stabilizer panels 214 (BK) and medial panels 206 (AK) may be allowed to rotate and/or swivel freely around their anchor points at or near the brim height—hence better self-aligning with the range of motion and contouring of the anatomy.

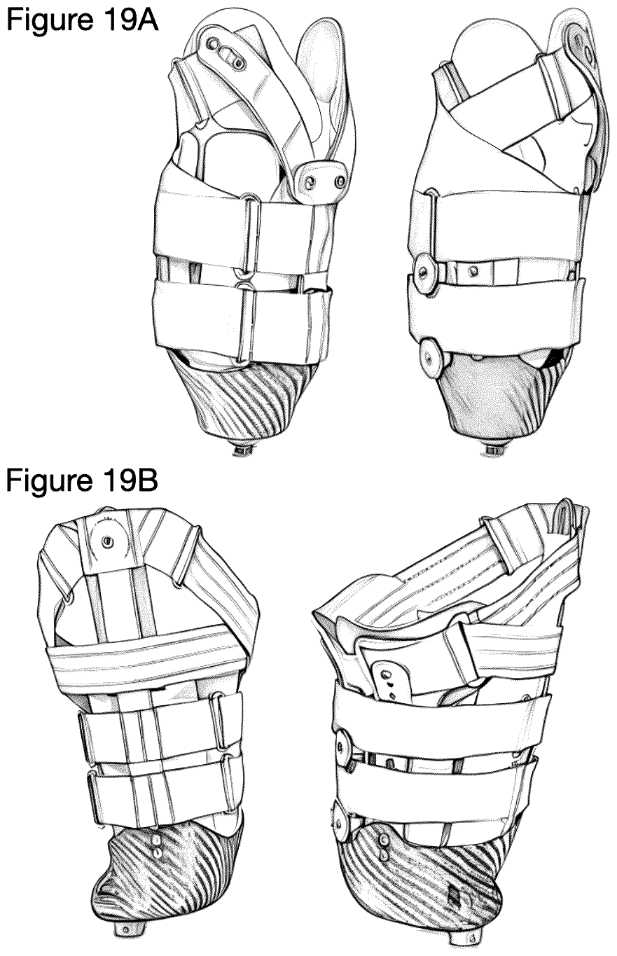

The modularity of this system may allow for a greater amount of the conventional rigid socket frame to be trimmed lower, and more of the socket's surface area to be replaced with conforming socket materials, as illustrated in FIG. 19A (BK) and 19B (AK). In FIGS. 18A and 18B, the conventional socket is used as the proximal mounting point for the fabric inner socket elements, by virtue of the unique trimming pattern of the conventional sockets frame. Conversely, in FIGS. 19A and 19B, only the distal end of the conventional socket's frame remains, and the proximal mounting points utilize the described modular frame structure connected to the carbon fiber base.

1. A prosthetic socket system for securing a residual limb, comprising:
(a) a flexible inner socket structure configured to conform to the shape of the residual limb, the flexible inner socket structure comprising:
(i) a plurality of modular conforming panels selectively adhered together in anatomically specific regions to form a composite flexible inner socket;
(ii) wherein at least a portion of the conforming panels are non-stretch; and
(iii) wherein the composite flexible inner socket comprises:
(A) portions that contact and cover a portion of the residual limb,
(B) portions that do not contact and cover portions of the residual limb,
(C) portions that are circumferentially connected around the residual limb,
(D) portions that are not circumferentially connected around the residual limb,
(E) portions that extend continuously in a longitudinal direction from a proximal end to a distal end of the residual limb, and
(F) portions that do not extend continuously in a longitudinal direction from the proximal end to the distal end of the residual limb;
(iv) wherein at least a portion of the composite flexible inner socket remains conforming after formation of the composite flexible inner socket;
(b) an external frame structure configured to provide structural support to the composite flexible inner socket and the residual limb during weight bearing, the external frame structure comprising:
(i) a distal end configured for modular connection to prosthetic foot or knee components;
(ii) a proximal end configured to attach and secure the composite flexible inner socket such that the composite flexible inner socket is at least partially suspended therefrom; and
(iii) wherein the external frame structure overlaps only a portion of the composite flexible inner socket;
(c) a circumferential containment system configured to conform to and control the shape of at least a portion of the composite flexible inner socket, the circumferential containment system comprising:
(i) at least one set of conforming straps positioned circumferentially around the composite flexible inner socket and the external frame structure;
(ii) an adjustor means operable to vary the tension of the conforming straps to effect selective circumferential tightening for secure containment of the residual limb within the composite flexible inner socket and external frame structure combination; and
(iii) wherein at least a portion of the conforming straps forms at least a portion of the prosthetic socket system's brim;
wherein the interplay between the outward forces exerted by the residual limb from within the prosthetic socket system and the inward forces resulting from the tension exerted by the circumferential containment system defines the anatomically specific socket shape and contouring of at least a portion of the composite flexible inner socket materials, and wherein the prosthetic socket system provides structural integrity to support weight bearing, biomechanical control of the residual limb during weight bearing, and anatomically appropriate contouring for comfortable ambulation.

2. The prosthetic socket system of claim 1, using an adjustor mechanism, comprising:
(a) a tensioning subsystem configured to variably adjust the tension applied to at least one containment strap, the containment strap comprising a webbing material;
(b) wherein the adjustor mechanism is operable to selectively wind or unwind the webbing material about a fixed rotational axis to modulate the applied tension; and
(c) wherein the adjustor mechanism incorporates a mechanical advantage—such as a gear assembly—to facilitate user-operated control of the webbing material's tension.

3. The prosthetic socket system of claim 1, wherein upon tightening of the adjustor mechanism, the composite flexible inner socket and residual limb are biomechanically pulled away from at least a portion of the external frame structure.

4. The prosthetic socket system of claim 1, wherein the plurality of modular conforming panels are interconnected using hook-and-loop (Velcro-compatible) fasteners.

5. The prosthetic socket system of claim 1, wherein the external frame structure further comprises a plurality of adjustable support extensions configured to accommodate variations in residual limb dimensions.

6. A prosthetic socket system for securing a residual limb, comprising:
(a) a flexible inner socket structure configured to conform to the shape of the residual limb, the flexible inner socket structure comprising:
(i) a plurality of modular conforming panels selectively adhered together in anatomically specific regions to form a composite flexible inner socket;
(ii) wherein at least a portion of the conforming panels are non-stretch; and
(iii) wherein the composite flexible inner socket comprises:
(A) portions that contact and cover a portion of the residual limb,
(B) portions that do not contact and cover portions of the residual limb,
(C) portions that are not circumferentially connected around the residual limb,
(D) portions that do not extend continuously in a longitudinal direction from the proximal end to the distal end of the residual limb;
(iv) wherein at least a portion of the composite flexible inner socket remains conforming after formation of the composite flexible inner socket;
(b) an external frame structure configured to provide structural support to the composite flexible inner socket and the residual limb during weight bearing, the external frame structure comprising:
(i) a distal end configured for modular connection to prosthetic foot or knee components;
(ii) a proximal end configured to attach and secure the composite flexible inner socket such that the composite flexible inner socket is at least partially suspended therefrom; and
(iii) wherein the external frame structure overlaps only a portion of the composite flexible inner socket;
(c) a circumferential containment system configured to conform to and control the shape of at least a portion of the composite flexible inner socket, the circumferential containment system comprising:
(i) at least one set of conforming straps positioned circumferentially around the composite flexible inner socket and the external frame structure;
(ii) an adjustor means operable to vary the tension of the conforming straps to effect selective circumferential tightening for secure containment of the residual limb within the composite flexible inner socket and external frame structure combination; and
(iii) wherein at least a portion of the conforming straps forms at least a portion of a prosthetic socket system's brim;
wherein the interplay between the outward forces exerted by the residual limb from within the prosthetic socket system and the inward forces resulting from the tension exerted by the circumferential containment system defines the anatomically specific socket shape and contouring of at least a portion of the composite flexible inner socket materials, and wherein the prosthetic socket system provides structural integrity to support weight bearing, biomechanical control of the residual limb during weight bearing, and anatomically appropriate contouring for comfortable ambulation.

7. The prosthetic socket system of claim 2, using an adjustor mechanism, comprising:
(a) a tensioning subsystem configured to variably adjust the tension applied to at least one containment strap, the containment strap comprising a webbing material;
(b) wherein the adjustor mechanism is operable to selectively wind or unwind the webbing material about a fixed rotational axis to modulate the applied tension; and
(c) wherein the adjustor mechanism incorporates a mechanical advantage—such as a gear assembly—to facilitate user-operated control of the webbing material's tension.

8. The prosthetic socket system of claim 2, wherein upon tightening of the adjustor mechanism, the composite flexible inner socket and residual limb are biomechanically pulled away from at least a portion of the external frame structure.

9. The prosthetic socket system of claim 2, wherein the plurality of modular conforming panels are interconnected using hook-and-loop (Velcro-compatible) fasteners.

10. The prosthetic socket system of claim 2, wherein the external frame structure further comprises a plurality of adjustable support extensions configured to accommodate variations in residual limb dimensions.

11. A prosthetic socket brim assembly, comprising:
(a) a frame structure;
(b) a plurality of conforming panels configured to form at least a portion of a brim;
(c) at least a portion of the panels being connected to or suspended from the frame structure;
(d) a fabric webbing component forming at least a portion of a brim of the prosthetic socket assembly, which is connected to at least a portion of the conforming panels; and
(e) an adjustment means operably coupled with the fabric webbing component, the adjustment means configured to selectively vary the tension of the fabric webbing component;
wherein selective tensioning of the fabric webbing component applies an inward force on the conforming panels such that the interplay between outward forces exerted by a residual limb and the inward forces resulting from the tension of the fabric webbing component defines an anatomically specific custom-fit brim shape and contouring of at least a portion of the conforming panels.

12. A method of fitting a prosthetic socket to a residual limb, comprising:
(a) modularly attaching a plurality of conforming panels—each panel having predetermined non-stretch and conformable regions and configured for hook-and-loop-compatible interconnection—in a selectively arranged manner based on the residual limb's anatomy to form a composite flexible inner socket having regions of tailored support, cushioning, and stretch properties, including contiguous areas designed to contact the limb and non-contiguous areas to permit ventilation and tissue movement;
(b) assembling an external frame structure about the residual limb, the external frame structure including a proximal attachment interface for suspending the composite flexible inner socket and a distal modular connection configured for attachment to prosthetic foot or knee components, wherein the frame structure aids in aligning the limb and supporting weight bearing;
(c) coupling the composite flexible inner socket to the external frame structure at a plurality of predetermined attachment points that secure the inner socket to the frame while maintaining its anatomical contour; and
(d) adjusting a tensioning mechanism associated with an integrated circumferential containment system—comprising one or more adjustable straps that form at least a portion of the socket's brim—to selectively tighten the composite flexible inner socket about the residual limb, thereby effecting a secure, custom-fit interface that dynamically conforms in real time to the unique contours and force distribution of the limb.

13. An external frame structure for a prosthetic socket system for securing a residual limb and supporting an anatomical knee joint, comprising:
(a) knee stabilizer components positioned medially and laterally around an anatomical knee to support the anatomical knee joint;
(b) at least one knee stabilizer component with an adjustment mechanism operably connected to the knee stabilizer, the adjustment mechanism configured to vary the angular orientation and/or positional placement of the knee stabilizer relative to the residual limb;
(c) an adjustment interface coupled to the knee stabilizer adjustment mechanism and operable by a user to set the knee stabilizer to an optimal alignment relative to the residual limb; and
(d) an integrated locking feature associated with the knee stabilizer adjustment mechanism, the locking feature configured to securely maintain the adjusted position of the knee stabilizer during weight-bearing activities without unintended movement.

14. The prosthetic socket system of claim 13, further comprising a conforming panel mounted to the knee stabilizer, wherein the conforming panel is formed of flexible material and is configured to dynamically contour to the residual limb as the socket is selectively tightened about the limb, thereby enhancing biomechanical knee stability and comfort.
